(19) United States
(12) Patent Application Publication
(10) Pub. No.: US 2024/0273815 A1
(43) Pub. Date: Aug. 15, 2024

(54) GENERATING SOUVENIRS FROM EXTENDED REALITY SESSIONS

(71) Applicant: Adeia Guides Inc., San Jose, CA (US)

(72) Inventors: Kamal J. Koshy, Aurora, CO (US); Dhananjay Lal, Englewood, CO (US)

(21) Appl. No.: 18/109,062

(22) Filed: Feb. 13, 2023

(51) Int. Cl. G06T 17/00; A61B 5/00; A61B 5/103; A61B 5/16

(52) U.S. Cl. CPC G06T 17/00; A61B 5/103; A61B 5/165; A61B 5/72

(57) ABSTRACT

Methods and systems are described for generating digital and physical artifacts and souvenirs. The generating is based on information generated during extended reality (XR) sessions including augmented reality, consumption of three-dimensional content, four-dimensional experiences, next-generation user interfaces, virtual reality, mixed reality experiences, and interactive experiences. Modules and sensors are provided for artifact generation. The modules and sensors may be interconnected to each other and to an XR device. The modules include an emotion module, a training module, and an artifact module. The methods and systems deliver improved artifacts for subsequent enjoyment of the XR experience. Artificial intelligence systems, including neural networks, are trained for improving the conversion. Models are developed for improving the conversion. Related apparatuses, devices, techniques, and articles are also described.

The present disclosure relates to automated content or object generation. Artifacts and souvenirs (digital or otherwise) are generated based on information generated during extended reality (XR) sessions including XR, augmented reality (AR), three-dimensional (3D) content, four-dimensional (4D) experiences, next-generation user interfaces (next-gen UIs), virtual reality (VR), mixed reality (MR) experiences, interactive experiences, and the like.

### BACKGROUND

Conventional approaches to souvenir generation in XR and gaming environments are limited. In one conventional approach, a mobile game includes fixed options to share, or record an image or video related to the game. Aside from routine user selection of game options, the format and content of an image or a video from the game for sharing is fixed.

In another conventional approach, players of XR games may configure desktop systems to share live game play content. The user may set up the XR session to include supplemental content during the XR session. Further modifications to the XR content require post-production editing of the video.

The conventional approaches are not configured to identify new images or scenes that are important to a particular user or tailored to specific user preferences. The conventional approaches do not account for variation of preferences for different users, a user's performance level, a user's emotions evoked by the gaming experience, or a user's real or XR surroundings. The conventional approaches are not easily configured for inclusion or exclusion of images of the user, inclusion or exclusion of playing characters and non-playing characters (NPCs), and the like.

As such, a need has arisen for methods and systems that overcome these problems and deliver improved generation of artifacts and souvenirs from XR and gaming sessions.

### SUMMARY

Methods and systems for generation of digital or physical souvenirs or keepsakes related to a user engaged in an extended reality session are provided. During the XR session, an emotional state of a user is determined or estimated. Also, a biomarker of the user may be detected. Collected or estimated information is analyzed to mark or predict an event of significance in the XR session. After a causal event is marked or predicted, sufficient information from the XR session is collected to generate the artifact or souvenir. XR headsets or glasses may be used to generate the initial content. The artifact or souvenir is generated in various digital and/or physical object formats. The content or object generation is automated. The content or object generation may be provided substantially on demand.

Various modules and sensors are provided for artifact generation. The modules and sensors may be interconnected to each other and to an XR device. The modules include an emotion module, a training module, and an artifact module. The emotion module is configured to receive and analyze information about a user, determine an emotion exhibited by the user (or a likelihood that the user is exhibiting the emotion), and output this emotional information to other modules. The training module is configured to incorporate inputs and outputs of various modules to generate a trained model for use in lieu of or in addition to the modules. The artifact module is configured to receive information from the modules and the device and generate an artifact in many formats (electronic and/or physical). One or more of the modules and sensors may be provided with the XR device or provided independently, for example, in a cloud-based system.

In some embodiments, the emotion module is configured to assess an emotional state of a first user or a first group of users relative to an emotional state of a second user or a second group of users engaged in a common XR experience. A significant shift in emotion (e.g., one team is happy while the other is sad) signals a causal event for capture of information to generate the artifact. In some embodiments, an array of multiple emotions is tracked and scored for one or more users. When scores cross over pre-determined thresholds, shift significantly, and/or exhibit significant positive or negative trendlines over a time period, such information may be utilized to signal the causal event. Throughout the specification, the term “significant” also refers to statistical significance in some embodiments.

User interfaces are provided for collecting information from a user or group of users prior to or after an XR session. In some embodiments, after a set point, the user may be prompted to authorize a curated list of artifacts based on the XR session. In some embodiments, before engaging in a new XR session, the user may be prompted to pre-authorize generation of the curated list. The user may also be pre-prompted to select types of scenes and/or particular emotional states for artifact generation.

In some embodiments, the artifacts present the XR session in a format matching the perspective of the user requesting the artifact. In other embodiments, the artifact may depict the user from a third-person perspective. The artifact may include a series of images or videos that capture an event of interest, before, during, and after a critical point in the XR session. All these types of artifacts may be combined in any suitable format or arrangement.

The present invention is not limited to the combination of the elements as listed herein and may be assembled in any combination of the elements as described herein.

These and other capabilities of the disclosed subject matter will be more fully understood after a review of the following figures, detailed description, and claims.

### BRIEF DESCRIPTIONS OF THE DRAWINGS

The present disclosure, in accordance with one or more various embodiments, is described in detail with reference to the following figures. The drawings are provided for purposes of illustration only and merely depict non-limiting examples and embodiments. These drawings are provided to facilitate an understanding of the concepts disclosed herein and should not be considered limiting of the breadth, scope, or applicability of these concepts. It should be noted that for clarity and ease of illustration these drawings are not necessarily made to scale.

The embodiments herein may be better understood by referring to the following description in conjunction with the accompanying drawings, in which like reference numerals indicate identical or functionally similar elements, of which:

FIG. 1A depicts a first user wearing an XR headset after losing an XR competition during a shared XR session, and a second user wearing an XR headset celebrating winning the XR competition, in accordance with some embodiments of the disclosure;

FIG. 1B depicts a group of users watching a souvenir video on a display, where the souvenir video is based on content generated during the XR session of FIG. 1A, in accordance with some embodiments of the disclosure;

FIG. 2 depicts a system including the XR headset of FIG. 1A, an external sensor, an emotion module, an artifact module, various forms of generated artifacts, and a training module, in accordance with some embodiments of the disclosure;

FIG. 3 depicts a first group of users, a second group of users, and the emotion module of the system of FIG. 2 configured to detect an emotional state of each user of the first and second group of users, in accordance with some embodiments of the disclosure;

FIG. 4A depicts a notification window for the XR headset of FIG. 1A, in accordance with some embodiments of the disclosure;

FIG. 4B depicts a notification window, in accordance with some embodiments of the disclosure;

FIG. 4C depicts a notification window, in accordance with some embodiments of the disclosure;

FIG. 5 depicts a device including a sensor pointed at a first user and a display device configured to display an image from the XR session, the image including a first avatar representing a second user and a second avatar representing a third user, in accordance with some embodiments of the disclosure;

FIG. 6 depicts a series of six souvenir images taken from a side view of a user in an XR space at points in time before, during, and after the user engaging in an event in the XR space, in accordance with some embodiments of the disclosure;

FIG. 7 depicts an emotional state of the user in FIG. 6 at each of the six points of the event and a capture event associated with the third point of the event, in accordance with some embodiments of the disclosure;

FIG. 8 depicts an emotional state of two users at each of six points of an event in a shared XR space and a capture event associated with the third point of the event, in accordance with some embodiments of the disclosure;

FIG. 9 depicts a first process for opting in a user, capturing content, detecting emotion, creating lists of content, post-processing content, and generating artifacts, in accordance with some embodiments of the disclosure;

FIG. 10 depicts a second process for opting in a user, capturing content, detecting emotion, temporarily storing content, permanently storing content, post-processing content, and generating artifacts, in accordance with some embodiments of the disclosure;

FIG. 11 depicts a third process for opting in a user, receiving content, analyzing content, determining an emotional state, determining whether a causal event for generating an artifact is occurring or is likely to occur, identifying session information, determining storage, generating an artifact, training a model, verifying the model, and transitioning to a subsequent session, in accordance with some embodiments of the disclosure;

FIG. 12 depicts subprocesses of the analyzing content of the third process of FIG. 11, in accordance with some embodiments of the disclosure;

FIG. 13 depicts first subprocesses of the determining of the emotional state of the third process of FIG. 11, in accordance with some embodiments of the disclosure;

FIG. 14 depicts second subprocesses of the determining of the emotional state of the third process of FIG. 11, in accordance with some embodiments of the disclosure;

FIG. 15 depicts third subprocesses of the determining of the emotional state of the third process of FIG. 11, in accordance with some embodiments of the disclosure;

FIG. 16 depicts an artificial intelligence system, which may be utilized with the system of FIG. 2 or FIG. 17, in accordance with some embodiments of the disclosure; and

FIG. 17 depicts a system including a server, a communication network, and a computing device for performing the methods and processes noted herein, in accordance with some embodiments of the disclosure.

The drawings are intended to depict only typical aspects of the subject matter disclosed herein, and therefore should not be considered as limiting the scope of the disclosure. Those skilled in the art will understand that the structures, systems, devices, and methods specifically described herein and illustrated in the accompanying drawings are non-limiting embodiments and that the scope of the present invention is defined solely by the claims.

### DETAILED DESCRIPTION

Emotion detection is connected to automatic content creation. For example, detection of an emotional state expressed by a user during a VR session signals generation of artifacts and related souvenirs. In some instances, artifacts are generated in near real time. In other instances, session information is stored for generation of artifacts at a later time. Sensors (e.g., cameras and biosensors) collect information about a user. For example, images of the user (e.g., the user's face) and/or biomarkers are analyzed to determine emotion during human interactions. The biomarkers include a heart rate, brain activity, and variations in circulation obtained with devices such as a pulse oximeter, an electroencephalogram (EEG), a photoplethysmogram (PPG), and the like. The sensor data are analyzed to determine an emotional state of the user. The determined emotions are an accurate predictor of user engagement and interest. In response to a determination of a relatively elevated level of engagement and/or interest, audiovisual input is captured, and content is generated based on the captured input. Information gleaned from a pool of users may inform the capturing. For example, specific locations (XR or otherwise) where users have commonly chosen to capture video and/or images are marked for automatic content generation in subsequent sessions. Also for example, specific points within the XR environment that commonly evoke strong emotion in a statistically significant number of users are marked for automatic content generation in subsequent sessions. Numerous examples are provided in detail below.

The generated souvenirs include works derived from the XR session such as curated images, rendered images, a video recap, a 3D model rendition in the XR setting, and the like. The generated souvenirs may serve as a keepsake of an experience or milestone. For example, a souvenir or memento is generated at an end of the XR session (e.g., winning a game, breaking a record, solving a puzzle, beating a competitor, or finishing a quest). Non-fungible tokens (NFTs) may be generated to mark an event of significance and optionally subsequently monetized by the user. The artifacts are generated without a need to spend significant time locating, selecting, and post-processing video and image artifacts. Generated souvenirs may be stored for future perusal. Content creators may, with user consent, utilize captured assets as promotional material. The content presented may include video snippets having images with or without post-processing. The content may be modified in post-processing to stylize the video snippets to include strong emotion displayed by the user. Stylized content may be generated, which includes emotion-specific stylization or content. For example, in response to detection of a “thrilled” user, a stylization might include superimposition of a phrase, “That was awesome!,” “Fantastic!,” and the like.

When the user is using an XR headset in the XR session, if sufficient storage is available, an entire session may be captured. The captured session may be post-processed. In some embodiments, session information is captured in response to detection of an emotional state satisfying a standard. The session information may include both scenes from a viewpoint of the user along with one or more third-party views that are generated by the XR system. The third-party views may not necessarily be part of the primary user's XR session. Information from sensors (e.g., cameras, infrared sensors, and the like) is captured. The sensor information may be captured and processed locally or remotely, e.g., in a cloud-based system.

An emotional state of the user may be detected and estimated using physiological measurements (e.g., heart rate, EEG, a change in a skin tone, eye movement tracking, pupil measurements, and the like) and machine learning systems. The machine learning systems may use sensor data as inputs. In multi-player or multi-user scenarios, the emotions of related users are tracked. In an online gaming scenario with multiple users on the same playing field, the emotions of the multiple users are tracked. An emotion detection engine (EDE) is configured to analyze the information for related users and perform statistical analysis (e.g., mean, mode, and the like) to detect an emotional sequence for groups of users. For example, a souvenir is generated for an entire team of users playing together to collaboratively beat an enemy team. In a collaborative game, positively correlated emotions of multiple users signal generation of souvenir content (e.g., image rendition, video recap, 3D model rendition, and the like). In a competitive game, negatively correlated emotions of multiple users signal generation of souvenir content. For example, in response to a determination that a first group of users have positively correlated emotions, e.g., emotions trending in the same direction, while a second group of users have emotions trending in an opposite direction, souvenir content is generated. In some competitive gaming environments, the user may be thrilled when some other particular user is removed from the playing field. That is, the removal of a particular user that evokes a thrill for one user may not do the same for a different user. A combination of a strong emotion for the one user and a weak emotion for another user may signal generation of artifacts and curated content. The generated content may be based on learned behavior from the preferences of multiple users.

If an entire sequence of an XR session is captured (including audiovisual information, random number seeds, player actions, NPCs actions, and the like), the XR session may be re-rendered from a different perspective (e.g., third-party perspective) at a later time. A time-stamped emotion sequence is correlated with the captured session information. The time-stamped emotion sequence and correlated session information are post-processed to generate a curated set of content to be presented to the user in different forms (e.g., image, video, and/or 3D content).

In another embodiment, selective storage and processing occurs. For example, the EDE is configured to signal the application in real time based on a thresholding process that determines whether a significant moment is occurring (or is about to occur). In response to a signal from the thresholding process, the application begins capturing content. For example, audiovisual information and 3D models may be captured using a scene descriptor format such as universal scene descriptor (USD), GL transmission format binary file (GLB), GL transmission file (glTF), immersive technology media format (ITMF), ORBX, and the like. Session information may be stored in a circular capture buffer that is continually overwritten. When the EDE signals that a significant moment is occurring (or is about to occur), the contents of the circular capture buffer may be stored, thereby also capturing the sequence leading up to the significant moment.

Action sequences may be detected. In response to detection of an action sequence, a viewpoint may be changed to a third-party viewpoint, so that an image of the user is included in the generated artifact or souvenir. In some embodiments, waypoints or markers in the extended reality space are included in the generated artifact or souvenir. The EDE may automatically perform the action sequence detection, generation of the third-party viewpoint, inclusion of waypoints or markers, and the like.

In some embodiments, a user is presented, via an interface, with selectable options for generating a souvenir. For example, for generating an NFT, the user may be presented with options for an image and/or a video to be included in the NFT. In another example, the user is presented with an option to print material including a 3D model of a selected scene from the XR session.

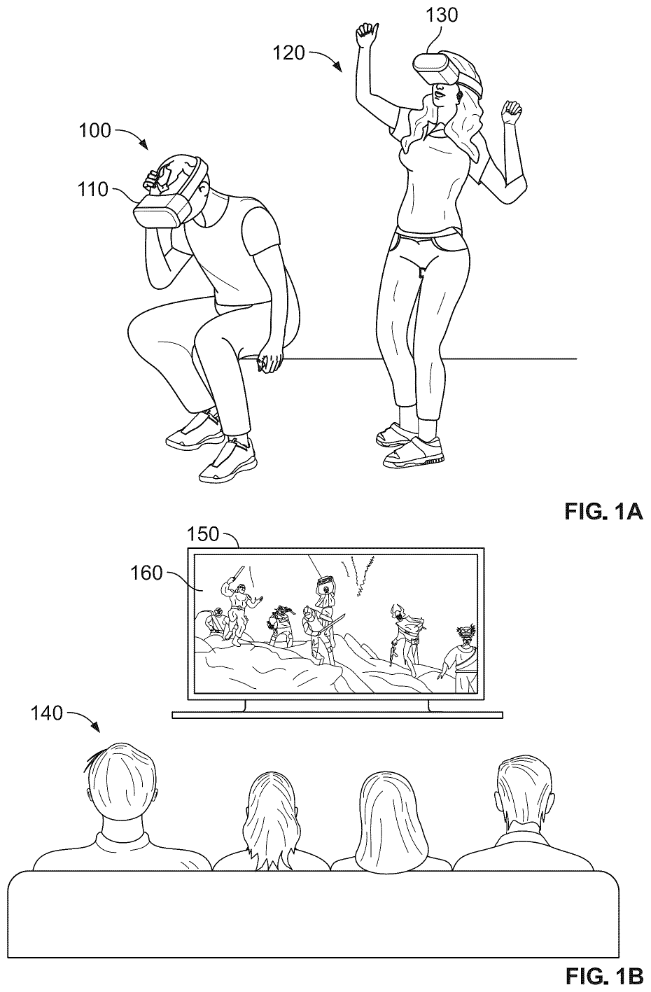

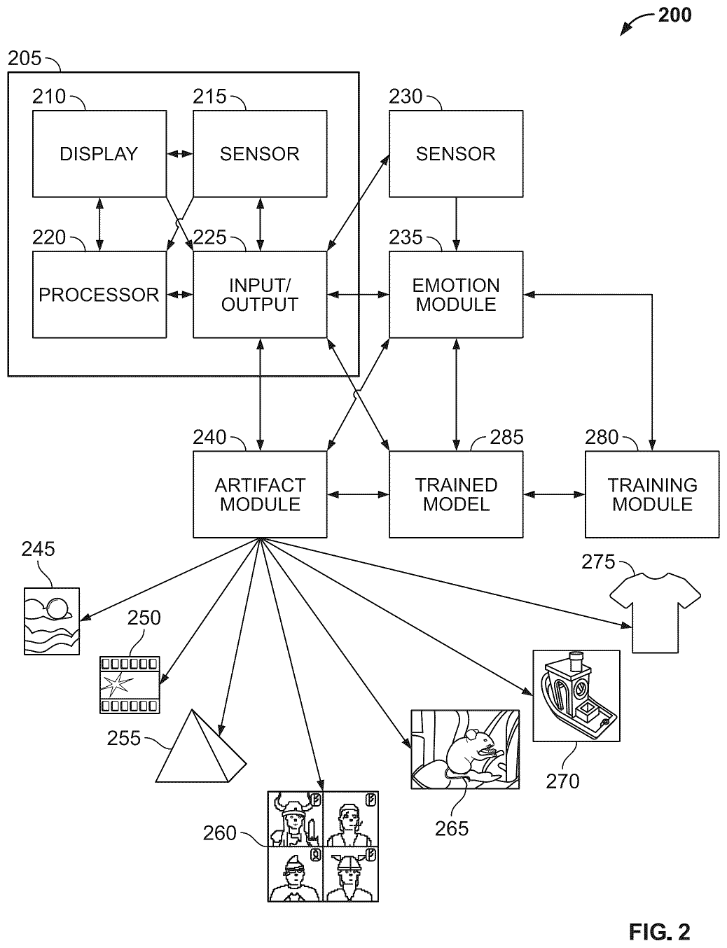

FIG. 1A depicts a first user 100 wearing an XR headset 110 after the first user 100 loses an extended reality competition during a shared XR session with a second user 120. The second user 120 is wearing an XR headset 130 and celebrating winning the XR competition. One or both of the headsets 110 and 130 may include one or more functions or structures of any of the devices of FIGS. 2, 5, 16, and 17, and the like.

The users 100 and 120 in FIG. 1A are depicted in the same physical space but may be participating in the competition in remote physical locations (not shown). The headsets 110 and 130 are shown with a similar head-mounted design but may have different configurations. Although the users 100 and 120 are depicted wearing the headsets 110 and 130, respectively, in accordance with the present disclosure, the present methods and systems are configured for similar functionality when the users 100 and 120 are engaging in an extended reality environment using other technologies such as projected environments, specially configured rooms, other types of wearable devices (e.g., gloves, suits, haptic devices, and the like), blended real-world/XR environments, combinations of these technologies, and the like.

FIG. 1B depicts a group 140 of four users watching a souvenir video 160 on a display 150, where the souvenir video 160 is based on content generated during the XR session of FIG. 1A. The souvenir video 160 includes a depiction of the second user 120 from a third-party perspective, where the second user 120 is celebrating the win in the XR environment (e.g., the avatar in the XR space is holding a boombox over its head). The souvenir video 160 may be configured for consumption on a conventional 2D display such as the display 150 of FIG. 1B. That is, the group 140 of users need not utilize XR equipment to enjoy the souvenir video 160. In other embodiments, a souvenir may be generated for consumption in an XR environment utilizing XR equipment such as the headset 110 or 130.

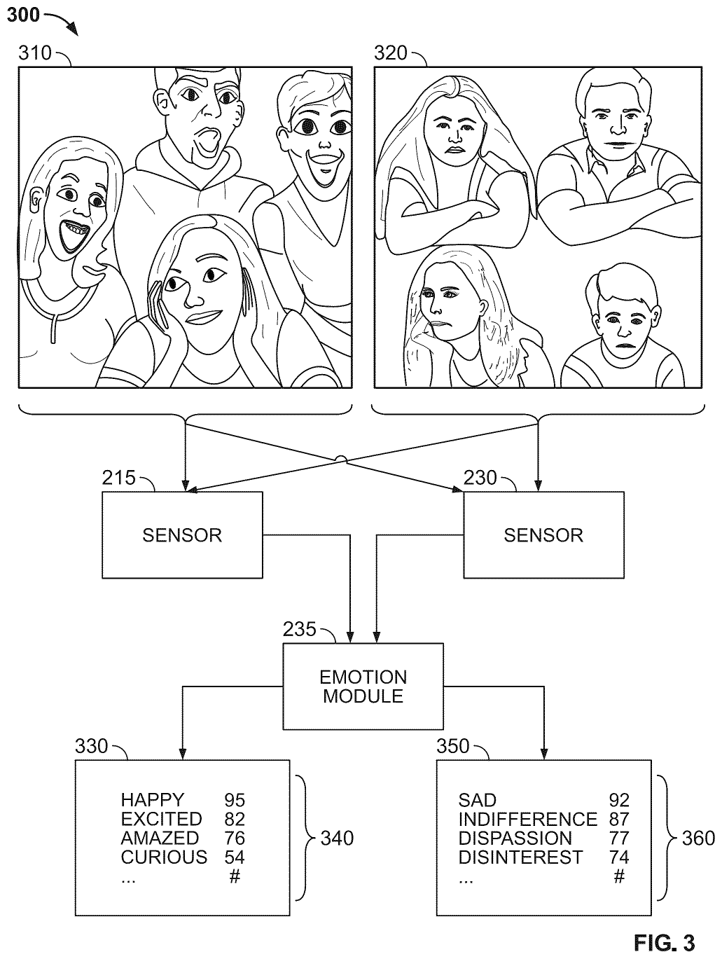

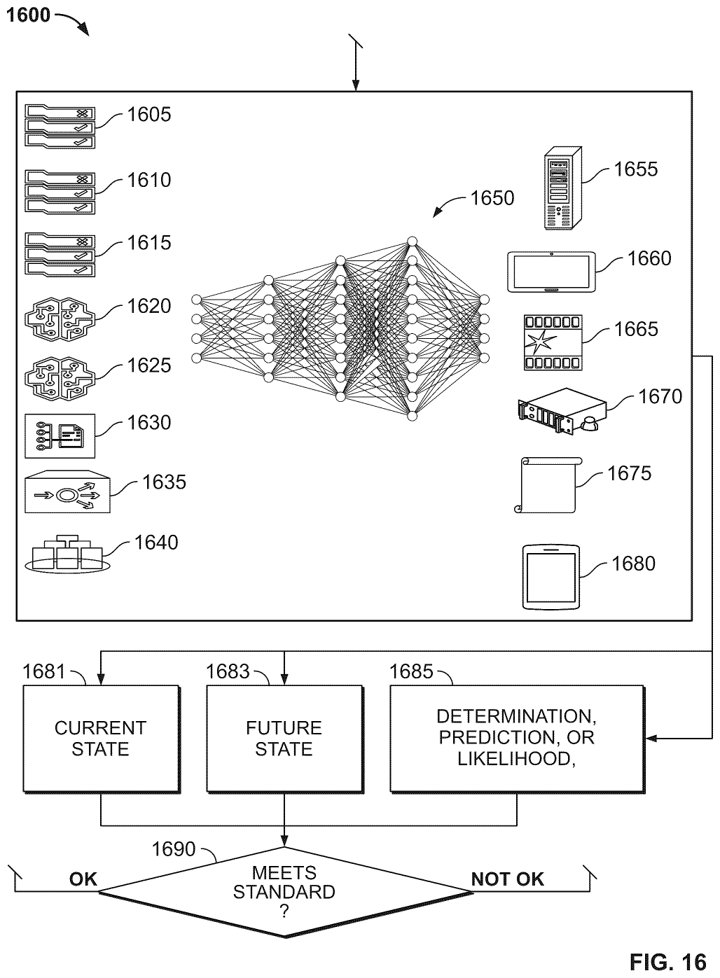

FIG. 2 depicts a system including various devices, modules, and types of artifacts. In some embodiments, a system 200 is provided with a device 205, an external sensor 230, an emotion module 235 (see, e.g., FIG. 3 and related descriptions), an artifact module 240, and a training module 280 configured to generate a trained model 285 (see, e.g., FIG. 16 and related descriptions). In FIG. 2, the system 200 is depicted with the device 205 being separate from the emotion module 235, the artifact module 240, and the training module 280. For instance, the training module 280 may be provided in a cloud-based system. However, the functionality of the emotion module 235, the artifact module 240, and the training module 280 may be incorporated into the device 205. The XR headset 110 or 130 of FIG. 1A may include the features of the device 205.

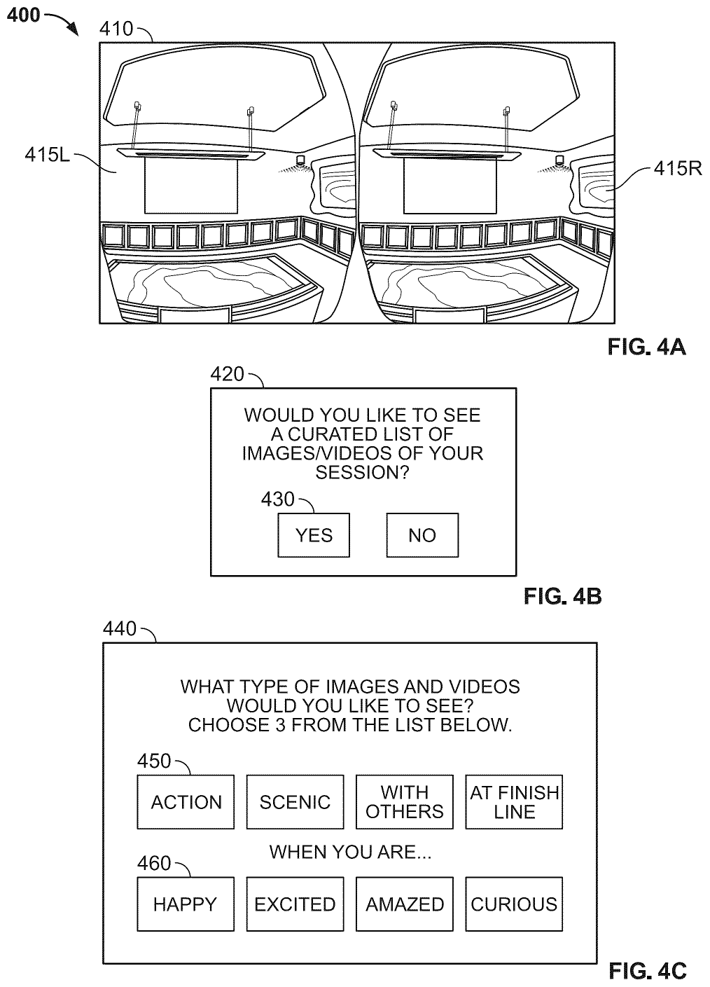

The device 205 may include a display 210, a sensor 215, a processor 220, and an input-output device 225. Each of the display 210, the sensor 215, the processor 220, and the input-output device 225 may be operatively connected within the device 205. In the embodiment of FIG. 2, the input-output device 225 is configured to send information out of the device 205 and receive information from external sources. The device 205 may communicate via wired or wireless communication systems with external devices. In some embodiments, the device 205 is a head-mounted display, and the display 210 includes a small optical display in front of one or both eyes of the user. For example, the display 210 may include a first display for a left eye of the user and a second display for a right eye of the user as shown in FIG. 4A. The input-output device 225 is configured to send or receive information from at least one of the external sensor 230, the emotion module 235, the artifact module 240, the training module 280, a combination of the same, and the like. The use of arrows in FIG. 2 and throughout the disclosure is not intended to be limiting. One-way, two-way, duplicate, parallel, and/or serial functionality may be added or deleted in any suitable combination.

Sensors are employed that are useful for detecting user emotion in an XR/AR/VR/MR session. Data from the sensors is captured and stored along with user session video and images. The data from the sensors is timestamped and correlated with the user session video and images. The sensors used to detect emotions may be different from those used for delivering the XR session. The sensors may include any suitable combination of audio, video, and image sensors. and the like. The data from the sensors may be preprocessed in the headset, in a local computing resource, and/or in the cloud. There may also be a pre-trained database for detecting emotion. The pre-trained database may be provided in the cloud as an initial starting point for a machine learning model to detect emotion.

One or more of the display 210, the sensor 215, and the external sensor 230 may be any suitable device for transmitting information, receiving information, transmitting sensations, and/or receiving sensations directly and/or indirectly to and/or from the user of the device 205 and/or an environment in which the user is physically located. In some embodiments, only the sensor 215 of the device 205 is required. In other embodiments, only the external sensor 230 of the system 200 is required. In still other embodiments, both the sensor 215 and the external sensor 230 are required. Although each of the sensor 215 and the external sensor 230 is referred to as a singular device, each may include an array of sensors in any suitable combination. One or both of the sensor 215 and the external sensor 230 may include one or more devices for transmitting and/or receiving a location of the user, audio information, visual information, a movement of a body part of the user, biological information, thermal information, haptic information, and the like. The device for transmitting and/or receiving the location of the user includes a six degree-of-freedom (6DoF) position sensor, a tracking sensor, a simultaneous location and mapping (SLAM) system, a global positioning system (GPS), a combination of the same, and the like. The device for transmitting and/or receiving audio information includes a speaker, a headphone, a directional microphone, a combination of the same, and the like. The device for transmitting and/or receiving visual information includes a display, an image sensor, a depth sensor, a front-facing camera, a user-facing camera, an ambient light sensor, an infrared sensor, a combination of the same, and the like. The device for transmitting and/or receiving a movement of a body part of the user includes an eye-tracking camera, a body-mounted sensor, an accelerometer, a gyroscope, a magnetometer, a combination of the same, and the like. The device for transmitting and/or receiving biological information includes a biosensor, digital holograph, electrode (e.g., attached to finger, palm, sole of the foot, chest, scalp, and/or limb), electroencephalograph, heart rate monitor, infrared camera (e.g., pointed towards one or both eyes), magnetic resonance sensor, microphone, near-infrared light sensor, optical camera (e.g., pointed toward face), optical sensor (e.g., attached to finger, toe, and/or earlobe), photoplethysmography device, pulse oximeter, a combination of the same, and the like. The device for transmitting and/or receiving thermal information includes a thermometer, a thermohaptic device, a Peltier heater, a thermoresistive heater, a thermosensor, a thermoresistive sensor, a pyroelectric sensor, a thermoelectric sensor, a combination of the same, and the like. The device for transmitting and/or receiving haptic information includes a piezoelectric device, a force feedback device, an air vortex device, an ultrasound device, a combination of the same, and the like. Information from one or more of the display 210 of the device 205, the sensor 215 of the device 205, and the external sensor 230 of the system 200 may be inputted into the processor 220, the emotion module 235, the artifact module 240, the training module 280, an external processor or circuitry, a combination of the same, or the like for processing.

The artifact module 240 is configured to receive information from any of the devices noted above, process the received information, and generate an artifact therefrom. The artifact 240 is configured to directly and/or indirectly generate at least one of an image 245, a video 250, a three-dimensional model 255, a non-fungible token (NFT) 260, a holographic image 265, a three-dimensional printed object 270, a piece of merchandise 275, a combination of the same, or the like. The content may be stored in any suitable 3D format. The artifact may be presented to the user, for example, as 3D digital art (e.g., a holographic display) or 3D printed material.

The generated artifact may represent information of emotional significance to the user in the XR environment. For example, in an XR game involving a quest to find a token, the generated artifact may be the three-dimensional printed object 270 in the form of the token found by the user in the quest of the game. The artifact module 240 is configured to generate instructions suitable for use by a 3D-printer sufficient to generate the object 270. Similarly, for generating the piece of merchandise 275, the artifact module 240 is configured to output a high-resolution image from the XR environment suitable for printing a graphic T-shirt as the piece of merchandise 275. That is, using the generated content, the user may be given an option to transfer the generated content to souvenirs such as T-shirts, cups, mugs, name plates, and the like. Other suitable artifact generations may be performed with non-limiting examples provided throughout the present disclosure.

In some embodiments, the processing for generating the artifact is performed external to the artifact module 240. That is, the artifact module 240 may be configured to generate minimally processed artifacts drawn from the XR session. Any of the generated artifacts may be utilized as a security or form of intellectual property. For example, when generating the NFT 260, and when the NFT 260 is intended to form a security or to be utilized for monetization, information about the NFT 260 may be sent to an appropriate compliance and/or registration system (not shown) for regulatory purposes, such as compliance with the U.S. Securities Exchange Act of 1934. The user may be presented with an option to generate the NFT for use in future play within the same XR/AR/VR/MR space.

The information from the system 200, e.g., information from the one or more sensors 215, 230, is provided for emotion detection. FIG. 3 depicts a first group 310 of users, a second group 320 of users, and the emotion module 235 of the system 200 of FIG. 2. The emotion module 235 is configured to detect an emotional state of each user of the first and second group 310, 320 of users. The emotion module 235 is configured to receive a biometric signal from a sensor (such as the sensors 215, 230), perform emotion detection techniques, determine features from the received information, and infer or predict a likelihood that a particular emotional, psychological or behavioral state is being experienced by the user. The techniques for emotion detection include at least one of electro dermal activity, electroencephalogram, eye-tracking, facial expression analysis, functional magnetic resonance imaging, functional near-infrared spectroscopy, heart rate variability (HRV), speech emotion recognition, a combination of the same, or the like. The biometric signal includes at least one of activity of facial muscles, change in skin conductance, changes in electrical activity of the brain, concentrations of oxygenated versus deoxygenated hemoglobin in blood or the blood vessels of the brain, corneal reflection, pupil dilation, variability in heart contraction intervals, voice, a combination of the same, or the like. The features determined from the received information include at least one of activation of peripheral input (e.g., a fire button on a game controller), blood oxygen level-dependent feature, EEG event-related potential, EEG frequency band power, EEG functional connectivity, eye blink, eye fixation, eye gaze, eye movement, eye saccades, head orientation, head position, HRV frequency domain, HRV non-linear domain, HRV time domain, pupil dilation, skin conductance response, skin phasic activity, skin tonic activity, voice prosodic feature, voice spectral feature, a combination of the same, or the like. The collected information is analyzed to infer (e.g., predict a likelihood) that a particular emotional, psychological or behavioral state is exhibited by the user at a particular point in time. The emotional, psychological or behavioral state includes at least one of admiration, adoration, amusement, anger, anxiety, appreciation, arousal, attention, awe, awkwardness, boredom, calmness, cognitive task engagement, compassion, confusion, contempt, contentment, craving, decision-making, depression, desire, disgust, drowsiness, embarrassment, engagement, entrancement, envy, excitement, fatigue, fear, fright, gratitude, grief, guilt, happiness, hope, horror, hunger, indifference, interest, jealousy, joy, love, memory, mental arithmetic engagement, mental workload, motor execution, nostalgia, pain, passion, pleasure, pride, rage, relief, romance, sadness, satisfaction, shame, stress, surprise, valence (i.e., pleasantness or unpleasantness of an emotional stimulus), visual attention, a combination of the same, or the like. In some embodiments, relatively simplistic groups of emotions are assessed, e.g., positive or negative, and/or a short list of emotional states such as anxiety, arousal, calm, fear, pleasantness, sadness, stress, and valence.

In the example of FIG. 3, sensors 215, 230 collect information from the first group 310 and the second group 320 right after the first group 310 played as a first team that won a competition in an XR space and the second group 320 played as a second team that lost the competition. The emotion module 235 analyzes the collected information to determine a score for each of a series of possible emotional states. The top emotional states may be determined and averaged for the group or analyzed for each user. In this example, the first group 310 is determined to be predominantly exhibiting four positive emotional states 340, i.e., happy, excited, amazed, and curious. The first group 310 is determined to have a happy score of 95 (out of one hundred), an excited score of 82, an amazed score of 54, and a curiosity score of 54. In some embodiments, the score for each emotion may be expressed as a part of 1.00 or percentages. Whereas, the second group 320 is determined to be predominantly exhibiting four negative emotional states 360, i.e., sad, indifference, dispassion, and disinterest. The first group 310 is determined to have a sad score of 92 (again out of one hundred), an indifference score of 87, a dispassion score of 77, and a disinterest score of 74. The number of emotional states need not necessarily be four. Any suitable number of emotional states may be monitored in any suitable combination. In some embodiments, in response to the emotion module 235 detecting an emotional state of a user at a score crossing a predetermined level, e.g., an emotional state score rising above a score of 50 on a scale from 0 to 100 (or above a score of 0.50 on a scale from 0.00 to 1.00), a subsequent action or signal (e.g., capturing an image of the face of the user, or an image of an avatar of the user in the XR environment) is performed. In some embodiments, an emotional matrix may include a number of emotions scored collectively up to a single maximum score of one hundred (or 1.00), and the like. Any suitable scoring system may be employed and need not necessarily be numeric or percentage based.

An XR game configured with the emotion detection functionality disclosed herein facilitates prompting of a user, in advance of the game or after the fact, to select from a curated list of content items available for artifact generation. For example, the XR session is captured and stored locally or in the cloud. Before or after the session, the user is prompted to select how the curated content should be presented to the user. Initially, user permission may be requested before further processing.

FIG. 4A depicts a notification window 400 for the XR headset of FIG. 1A. In this example, each of the XR headsets 110, 130 includes the display 210, which is configured to present the first display for the left eye of the user and the second display for the right eye of the user as shown in FIG. 4A. The display 210 is further configured to generate a left side image 415L and a right side image 415R, which the user perceives as a single 3D image 410.

FIG. 4B depicts a notification window 420, which prompts a user to select from user selectable options 430 to opt-in or opt-out of generation of the curated list. With user permission (i.e., user selection of Yes among the options 430), the user is presented with a list of themes from which to choose (FIG. 4C). FIG. 4C depicts a notification window 440, which prompts a user to select from a first group of user selectable options 450 and a second group of user selectable options 460. In this example, the user is asked to choose a number of types of scenes 450 (e.g., action, scenic, with others, and at the finish line) and a number of types of emotions 460 (e.g., happy, excited, amazed, and curious).

The list of options includes presentation of content identified by machine learning, artificial intelligence, and emotion-based systems. For example, the artificial intelligence system is configured to determine that a scene in a game is at least one of action, scenic, with others, at finish line, a combination of the same, or the like. The emotion-based system is configured to determine that one or more users is likely to have an emotional state that is positive or negative. In some embodiments, the system may determine that a user is exhibiting an emotional state that includes at least one of happy, excited, amazed, curious, sad, indifferent, dispassionate, or uninterested, a combination of the same, or the like (see, e.g., FIG. 3). The scene, video, or 3D representation of the scene may be analyzed with a combination of machine learning, artificial intelligence, and image processing. User emotion may be determined by analyzing information from “real world” sensors to estimate an emotional state of the user. That is, user emotion is generally not captured by capturing scenes, video, or images from the XR space. Post-processing may be required to determine user emotion. The machine learning process may be personalized with user-specific information including demographic information. The demographic information includes at least one of age, gender, nationality, language preference, cultural group, group membership, team membership, a combination of the same, or the like. The personalized information may be used to augment the accuracy of emotion detection with the machine learning process.

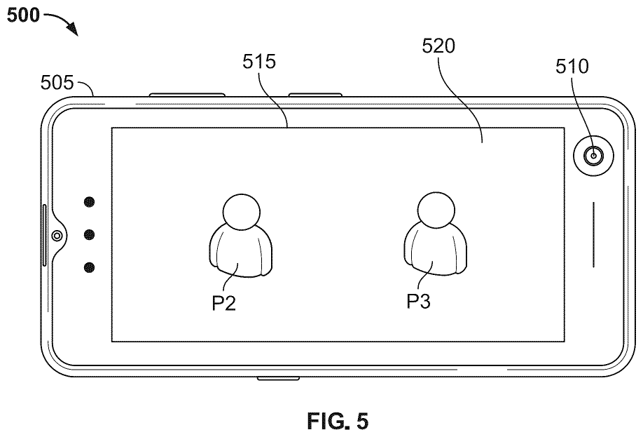

In some embodiments, a device is configured to capture raw information about the user. FIG. 5 depicts a device 500 having a housing 505 including a sensor 510 in the housing 505. The sensor 510 is pointed at a first user (not shown). The housing 505 of the device 500 includes a display device 515 configured to display an image from the XR session to the first user. That is, the sensor 510 and the display device 515 are operationally oriented in the same general direction (e.g., out of the screen). The sensor 510 may include one or more of the sensors described herein (e.g., sensors 215, 230). In this example, the image includes a first avatar P2 representing a second user and a second avatar P3 representing a third user. The first, second, and third users are engaging each other in a common XR session. The image may include depiction of an XR environment 520. Although a single image for display is shown in FIG. 5, a left/right 3D image display system such as that shown in FIG. 4A may be provided.

The device 500 may be an XR/AR/VR/MR headset with images and videos shown on a screen. The images and videos generated during the XR session may be analyzed and used to detect which scenes of the XR session are action scenes, scenic scenes, scenes with others, scenes at waypoints, and the like. The waypoints may include a finish line and other important session locations.

The device 500 may include a camera 510 and/or an array of sensors pointing at the user. The camera and/or sensors may be configured to capture information including images, videos, and physiological readings (e.g., heart rate, skin color change detection, brain activity, respiration rate, and the like). The captured information may be post-processed to detect user emotion as described herein. The EDE, which may include the emotion module 235, may be configured with local processing in a headset or processing may be performed remotely, e.g., with cloud computing.

The device 500 may include physiological sensors (e.g., a heart rate monitor, a smart watch, an EEG, a respiration rate sensor, and the like). The physiological sensors may be used along with camera information to augment accuracy of emotion detection.

Historical information of the user may be stored and accessed. The historic information may be used to verify accuracy of emotional detection. For example, historic heart rate and skin tone readings may be compared with real-time readings to determine that a user is excited or happy relative to past information.

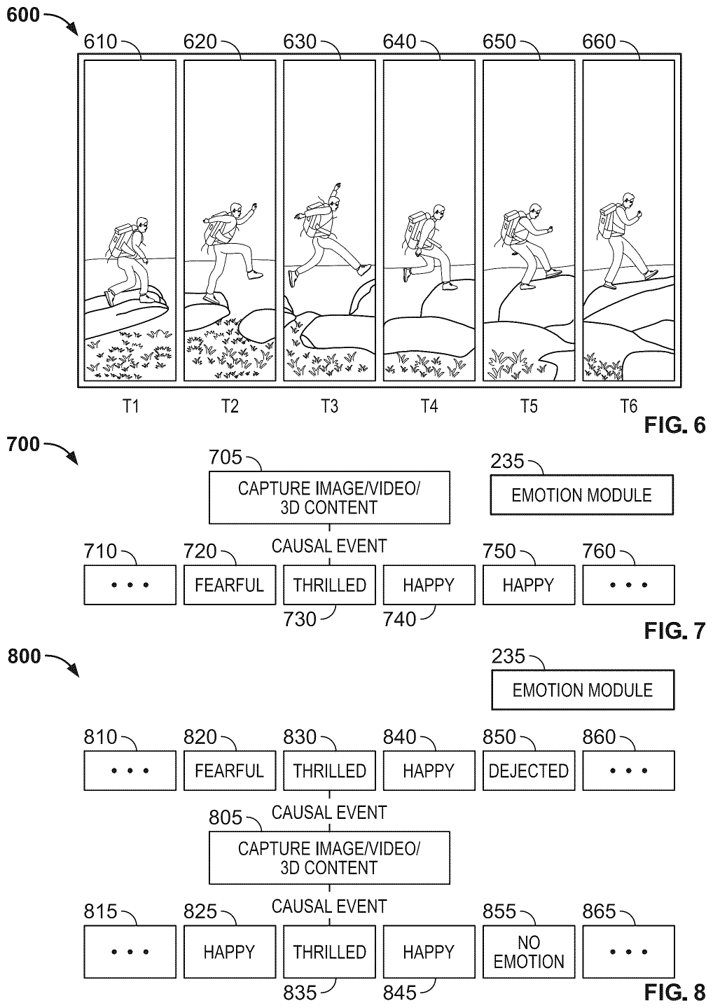

Examples of processing of image, video, and/or 3D content in conjunction with emotion detection are depicted in FIGS. 6-8, inclusive. The EDE detects emotion by processing information captured from one or more sensors (e.g., a camera and a heart rate monitor). In some embodiments, a machine learning process detects the emotion. From the sequence of captured image, video, and/or 3D content, a subset of curated images and videos is presented to the user at the end of the session.

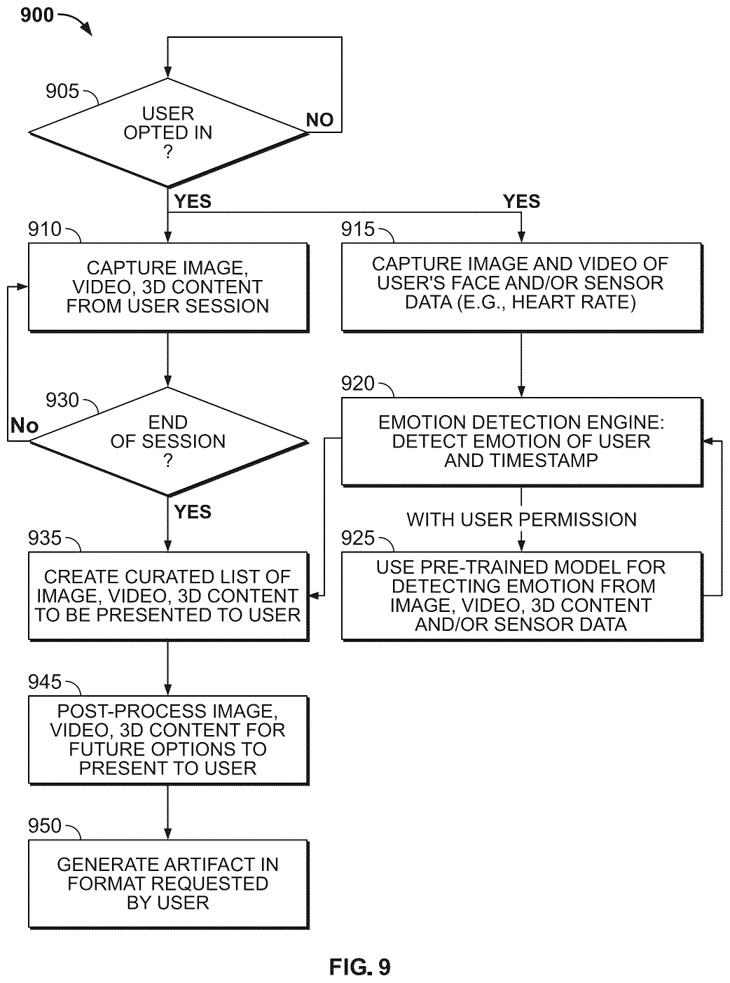

In one method, an entire user session is captured and timestamped. Sensors to detect emotions capture timestamp information. After emotion detection, a set of curated content is generated based on the user emotion. In another method, a fixed size buffer is used, and gross level emotion detection is performed locally, e.g., within a head-mounted display (HMD), or in a local computing device. Information is captured only at interesting points in the session, for example, when a gross level EDE detects a particular emotion. Curated content may be fine-tuned via cloud-based processing systems. Once the emotion is detected, the detected emotion may be correlated with the timestamped user session. Using the timestamped user session correlated with the detected emotion(s), content is curated and presented to the user. In some embodiments, changes in emotion signal curation. For example, detecting a change in emotion from thrilled to happy (e.g., in FIGS. 7 and 8) signals capture of the user session and generation of the artifact. In some embodiments, the user is presented with specific moments they would like to capture, and, with these selections, the system monitors for emotions that match user preferences. In some instances, the user may be presented with content that does not match the user preferences in order to test whether the user might be interested in other emotions or modalities.

FIG. 6 depicts a series of six souvenir images 610, 620, 630, 640, 650, 660 taken from a side view of a user in an XR space taken at six times, i.e., at times t1, t2, t3, t4, t5, and t6, respectively, that occur before, during, and after the user is engaging in an event in the XR space. In this example, a detected emotion of “thrill” signals the system to capture session information for generation of an artifact 600 including a combination of the side view images 610-660, inclusive. The artifact 600 does not, in this example, depict the user's first-person perspective in the XR environment (which might be considered less interesting as a keepsake), but instead changes the viewpoint to that of a third party viewing the user in the XR environment including the XR chasm below the user.

FIG. 7 depicts a process 700 of detecting an emotional state of the user in FIG. 6 at each of the six points 710, 720, 730, 740, 750, 760, respectively, of the event, and a capture event 705 associated with the third point 730 of the event. The emotional state is determined by the emotion module 235. The capture event 705 may include image, video, and/or 3D content. In this example, the detected emotional state at the first and sixth points 710 and 760 may be indefinite or close to an average for the user (represented with an ellipsis). At the second point 720, immediately prior to making a leap over a chasm, the emotion module 235 detects “fearful” as the user's likely emotional state. At the third point 730, when the user is mid-air over the chasm, the emotion module 235 detects “thrilled” as the user's likely emotional state, which, in this example, signals the capture event 705. The capture event 705 includes content before and after the causal event (e.g., two images before, one during, and three after the causal event). At the fourth point 740, when the user has one foot on solid ground past the chasm, the emotion module 235 detects “happy” as the user's likely emotional state, which continues to the fifth point 750.

In the case of multi-user scenarios, additional optimizations may be performed to augment the curated and generated content. An example of a process 800 for multi-player user emotion capture is shown for a two-player scenario in FIG. 8. In the process 800, an emotional state of a first user is captured at each of six points 810, 820, 830, 840, 850, and 860 during an event in an XR space shared with a second user, and an emotional state of the second user is captured at each of six corresponding points 815, 825, 835, 845, 855, and 865, respectively, during the same event in the shared XR space. A capture event 805 associated with the third point 830, 835 of the event in which both the first user and the second user are determined to be exhibiting a “thrilled” emotional state. Multi-player emotions may be captured to generate curated content of group activities. In addition, multi-player emotions may be used to capture common sessions for further post-processing. Emotions from other users may be used to signal the capture of the user session, for example, when the user is not displaying a decipherable emotion (e.g., at the point 855 where “no emotion” is determined).

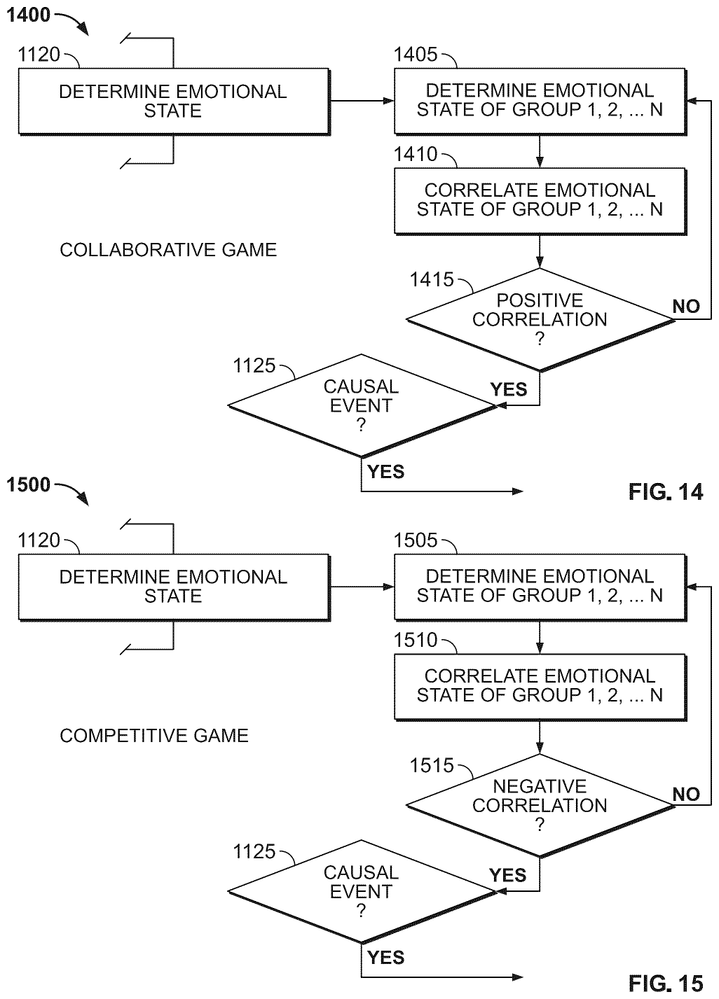

Variations for competitive and cooperative (or collaborative) scenarios may be performed. In the competitive scenarios (see, e.g., FIG. 15), capture may be signaled when one user is determined to be happy and another user is determined to be dejected (see, e.g., FIGS. 1A and 3). In the cooperative scenarios, user content generation may be signaled when multiple people are happy (e.g., the first group 310 of FIG. 3), and group content capturing may be signaled (see, e.g., FIGS. 8 and 14). For the competitive scenarios, the EDE may detect when a single user (or users in the same team) has positive emotions and users on a different team (or competitive users) have negative emotions (e.g., FIG. 3). That is, a negative correlation of emotions occurs when emotions of subject users move in opposite directions. For the cooperative scenarios, the EDE may detect a positive correlation of emotions. That is, emotions are positive and move in the same direction for multiple users or users in a team. In an XR reality setting, there may be occasions for applying cooperative scenarios of emotion detection and competitive scenarios of emotion detection.

Options for emotion processing are summarized in Table 1 below.

TABLE 1
Options for Emotion Processing

| | Single player scenario | Multi-player scenario |
|---|---|---|
| Limited storage (e.g., HMDs) | Gross level emotion detection | Same as the single player |
| and limited networking | in the HMD, with some | scenario. Also, utilize |
| | curation in the HMD. Fine- | competitive vs. cooperative |
| | tuned curated content | scenarios to generate curated |
| | generated by processing the | content. |
| | reduced set of content | |
| | generated by the HMD with | |
| | local emotion processing. | |
| Unlimited storage and low | Content stored in complete | Same as the single player |
| cost storage | form and processed to | scenario. Also, utilize |
| | generate final curated | competitive vs. cooperative |
| | content. | scenarios to generate curated |
| | | content. |

That is, the HMD or a local computing resource may hold a buffer of a user session. Emotion detection at a gross level may be performed in the HMD or the local computing resource. Once an emotion is detected (beyond a predefined threshold), capture of the user session is continued until, for example, a change of the emotion is detected. As such, this emotion-by-emotion storage sequence may reduce an amount of user session information requiring capture and storage. Post-processing of captured vignettes may be performed to improve an accuracy of emotion detection and to improve a quality of the curated list presented to the user.

The user session may be stored as a full session or in smaller segments. The storage may be performed in any suitable format. The format may be a scene descriptor format such as universal scene descriptor (USD), GL transmission format binary file (GLB), GL transmission file (glTF), immersive technology media format (ITMF), ORBX, and the like. Sufficient user session information may be captured to allow subsequent replaying of audiovisual content or images.

In addition to the emotion detection, single player scenarios, and multi-player scenarios noted above, image, video, and/or 3D content processing may be used to further augment the generation of curated content. Curated content may be modified by changing from a first-person view (e.g., FIG. 4A) to a third-person view (e.g., FIGS. 1B and 6). That is, with the third-person view, artifact content is generated where the user is seen in the content (rather than capturing the content from the user's relatively limited viewpoint). Waypoints and game checkpoints (e.g., level completion in a game) may be used to augment the generation of curated content. The game or user session may include pre-determined checkpoints or learned checkpoints where multiple users have responded positively to generated content. The checkpoints may include a finish line, achieving a goal, finishing a race, finishing a game level, and the like. User gaze and focus may be used to generate curated content along with content generated by emotion detection. The user gaze and the user focus may be used to identify areas of interest to the user.

Image, video, and/or 3D content is processed in accordance with a process 900, a process 1000, and/or a process 1100. Additional subprocesses 1200, 1300, 1400, 1500, and/or 1600 are provided. In some embodiments, the processing utilizes a pre-trained model or database. Processing may be performed with the EDE or the emotion module 235. In some embodiments, all content is stored and post-processed. Curation of content may be performed offline with content from the EDE or the emotion module 235.

FIG. 9 depicts a first process 900. The process 900 may include opting in a user, capturing content, detecting emotion, creating lists of content, post-processing content, and generating artifacts. The process 900 includes an opt-in process 905. In response to the user opting in (“Yes” at step 905), the process 900 continues with capture 910 of image, video, and/or 3D content from a user session. The capture 910 continues (“No” at step 930) until the session ends (“Yes” at step 930).

Also in response to the user opting in (“Yes” at step 905), the process 900 continues in parallel with capture 915 of images and/or video of a face of the user and/or sensor data (e.g., a heart rate of the user). After the capture 915, the process 900 continues to detect 920, using the EDE, an emotion of the user and to timestamp the same. After the detecting 920, the process 900 continues, with user permission, to use 925 a pre-trained model for detecting emotions from the image, video, 3D model, and/or sensor information. Output of the pre-trained model may be sent back for the detecting 920.

After the session ends (“Yes” at step 930) and/or after the detecting 920, the process 900 continues to create 935 a curated list of image, video, and/or 3D content to be presented to the user. After the creating 935, the process 900 continues to post-process 945 image, video, and/or 3D content for future options to present to the user. After the post-processing 945, the process 900 continues to generate 950 an artifact in a format selected by the user. The format includes, for example, at least one of the image 245, the video 250, the three-dimensional model 255, the NFT 260, the holographic image 265, the three-dimensional printed object 270, the piece of merchandise 275, a combination of the same, or the like.

A streamlined process is provided when devices and local computing cannot support storage, processing, and transmission sufficient to capture all of the generated content. To reduce demand on storage, processing, and transmission, gross level emotion detection is performed locally, e.g., within the HMD. In some instances, computing is performed outside of the HMD based on gross level content generated and/or curated within the HMD.

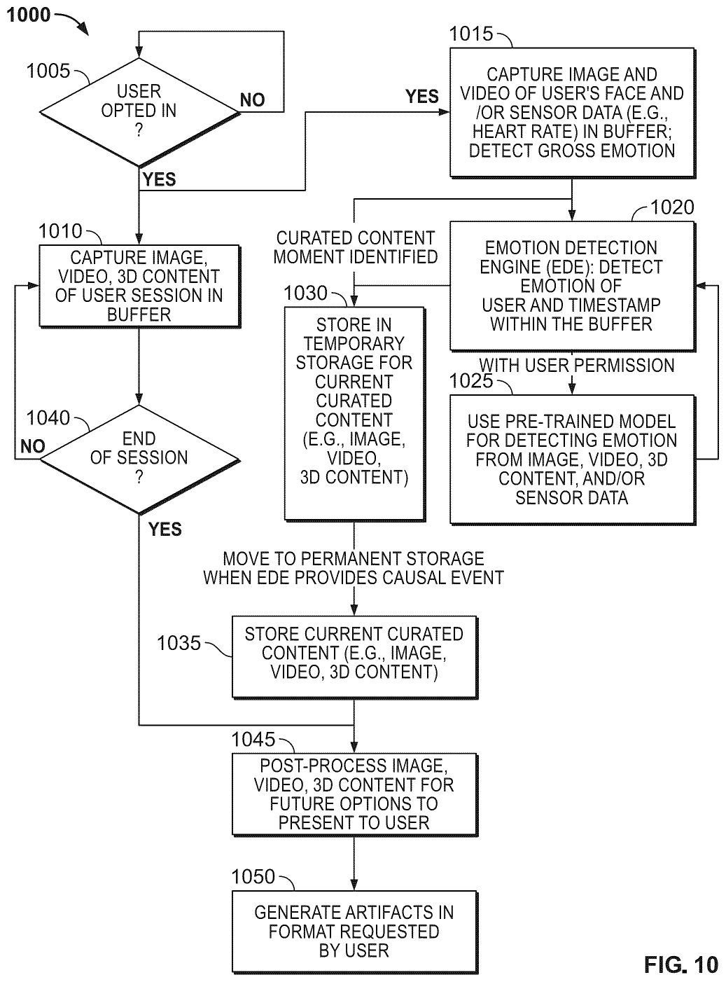

FIG. 10 depicts a second process 1000 for opting in a user, capturing content, detecting emotion, temporarily storing content, permanently storing content, post-processing content, and generating artifacts. The second process 1000 is generally similar to the first process 900 with like steps identified with the same last two digits between the two processes, e.g., steps 910, 915, 920, 925, 930, 945, and 950 are generally similar to steps 1010, 1015, 1020, 1025, 1030, 1045, and 1050, respectively, except as noted. Otherwise, descriptions of like steps are omitted for brevity.

The capturing 1015 of the second process 1000 is stored in a buffer and gross emotion detection is performed. The detecting 1020 and timestamping also occur in the buffer. After the capturing 1015 and/or the detecting 1020, the process 1000 continues, with identification of a potential curated content moment, to store output from the capturing 1015 and/or the detecting 1020 in a temporary storage for current curated content (e.g., image, video, and/or 3D content). After the EDE provides a signal marking a significant event (e.g., FIGS. 6-8, inclusive), the process 1000 continues to store 1035 current curated content (e.g., image, video, and/or 3D content) from the temporary storage in a permanent storage. The process 1000 continues in a manner similar to that described with respect to FIG. 9.

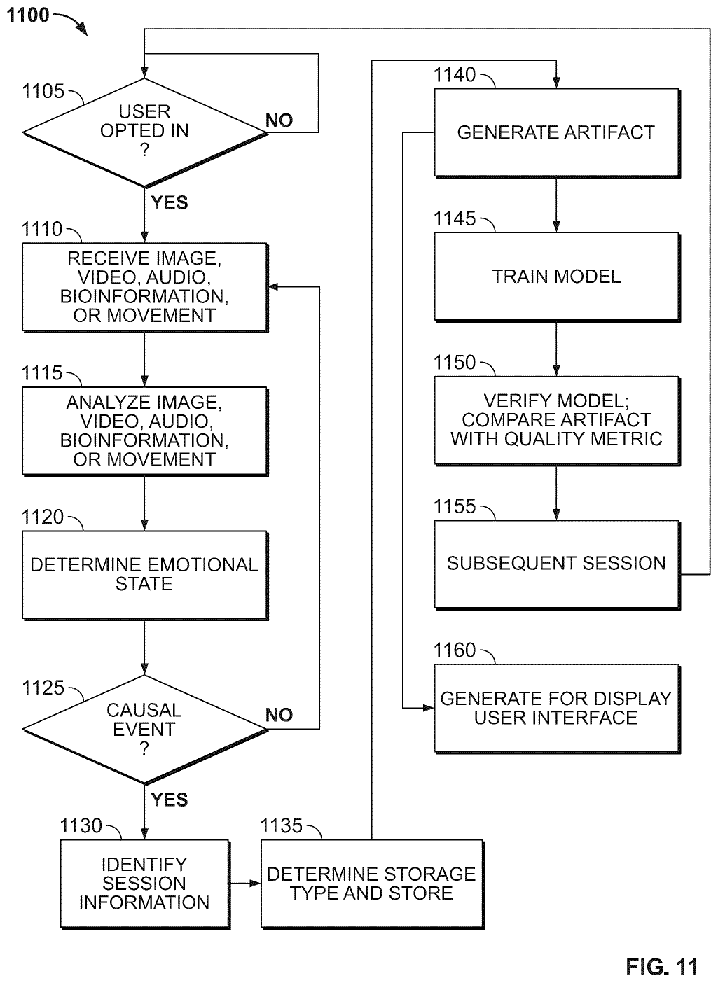

FIG. 11 depicts a third process. The third process may include opting in a user, receiving content, analyzing content, determining an emotional state, determining whether a causal event for generating an artifact is occurring or is likely to occur, identifying session information, determining storage, generating an artifact, training a model, verifying the model, and transitioning to a subsequent session. A process 1100 is provided for automatically generating a digital or physical souvenir or keepsake of a user engaged in an XR session. The process 1100 may include receiving 1110 at least one of an image of the user, a video of the user, an audio file of the user, bioinformation of the user, or a movement of the user. The process 1100 may include opting in 1105 a user. The process 1100 may include analyzing 1115 the at least one of the image, the video, the audio file, the bioinformation, or the movement. The process 1100 may include determining 1120 an emotional state of the user based on the analyzing 1115. The process 1100 may include identifying 1125 a causal event based on the determining 1120. The process 1100 may include in response to the identifying 1125 of the causal event (“Yes” at step 1125), identifying 1130 session information of the XR session. The process 1100 may include determining 1135 a type of a storage and storing information based on the determining 1135. The process 1100 may include generating 1140 the artifact based on the session information.

The term “causal event” may be used to describe any type of a signal for capture of information. The causal event may correspond directly with detection of a particular emotion. The causal event may correspond with a score representing an emotional state crossing over a pre-determined threshold, shifting significantly relative to an earlier status quo level, and/or exhibiting a significant positive or negative trendline over a given time period.

The process 1100 may include training 1145 a model with at least one of the image, the video, the audio file, the bioinformation, the movement, the XR session, the emotional state, the causal event, the session information, or the artifact. The process 1100 may include, during a subsequent XR session 1155, performing at least one of the receiving, the analyzing, the determining, the identifying of the causal event, the identifying of the session information, or the generating based on the model. The process 1100 may include verifying 1150 the model by comparing the artifact with a quality metric. The model may be trained with each of the image, the video, the audio file, the bioinformation, the movement, the XR session, the emotional state, the causal event, the session information, and the artifact. The receiving 1110 may include the image, the video, the audio file, the bioinformation, and the movement.

The model may be calibrated. The calibration may include prompting the user to display an emotion. The calibration may include capturing images of multiple users at a predetermined time in point in game play or a user session.

The analyzing 1115 may include the image, the video, the audio file, the bioinformation, and the movement. The bioinformation may include at least one of a heart rate, an electroencephalogram reading, or a respiration rate. The analyzing 1115 may include determining at least one of a change in a skin tone of the user, a blink rate of the user, or an eye movement of the user.

The determining 1120 of the emotional state of the user may include determining the emotional state of each user of a group of users including the user. Each user of the group of users may be engaged in the XR session.

The identifying 1125 of the causal event may include comparing the emotional state of each user of the group of users engaged in the XR session.

The causal event may occur in response to a significant shift in the emotional state of a first user of the group of users relative to the emotional state of a second user of the group of users. As used in this context, the term “significant shift” refers to a shift of statistical significance, meaning the observed shift in emotional state is unlikely to be explained by chance or random factors. In some embodiments, statistical significance may be indicated by a p value of about 0.05 or less.

In some embodiments, the XR session includes a collaborative game (e.g., FIG. 14), and the determining 1120 includes determining 1405 and correlating 1410 the emotional state of a portion of the group of users, and, in response to a positive correlation of the correlated emotional state of the portion of the group of users (“Yes” at step 1415), identifying the causal event (“Yes” at step 1125). In other embodiments, the XR session includes a competitive game (e.g., FIG. 15), and the determining 1120 includes determining 1505 and correlating 1510 the emotional state of a portion of the group of users, and, in response to a negative correlation of the correlated emotional state of the portion of the group of users (“Yes” at step 1515), identifying the causal event (“Yes” at step 1125).

The generating 1140 of the artifact includes generating at least one of an image 245, a video 250, a three-dimensional model 255, a non-fungible token (NFT) 260, a holographic image 265, a three-dimensional printed object 270, a piece of merchandise 275, a combination of the same, or the like. The process 1100 may include generating 1160 for display a user interface including a user selectable option to generate the artifact. The user interface may include options to generate the at least one of the image 245, the video 250, the three-dimensional model 255, the NFT 260, the holographic image 265, the three-dimensional printed object 270, the piece of merchandise 275, a combination of the same, or the like.

The process 1100 may include session information including a time-stamped emotion sequence (e.g., FIGS. 6-8). The time-stamped emotion sequence may be correlated with the session information. The session information includes information that may be provided in a scene descriptor format. The artifact may include a portion of the session information in the scene descriptor format. The session information may be stored in a circular capture buffer. The artifact may be generated based on the session information stored in the circular capture buffer.

The process 1100 may include detecting a type of sequence during the XR session. The type of sequence may be at least one of an action sequence, crossing a waypoint, crossing a marker, winning a competition, winning a game, or completing an action (see also, FIG. 4C re action, scenic, with others, and at the finish line).

The generating 1140 of the artifact may include changing a viewpoint of the XR session to include an image of the user (e.g., FIG. 1B or FIG. 6).

The process 1100 may include generating for display a user interface including a user selectable option to generate a curated list of content from the XR session (e.g., FIG. 4B). The process 1100 may include generating for display a user interface including user selectable options to identify conditions for generating the artifact. The conditions may include a scene type, and the emotional state of the user (e.g., FIG. 4C). The scene type may include at least one of action, scenic, with others, or at a finish line, and the emotional state includes at least one of happy, excited, amazed, or curious (e.g., FIG. 4C).

The artifact may include a series of content items at a time before, during, and after the causal event (e.g., FIG. 6).

The process 1100 may include determining a limited or unlimited storage state of a storage device configured to store at least a portion of the XR session and adjusting an amount of the session information based on the determining of the limited or unlimited storage state.

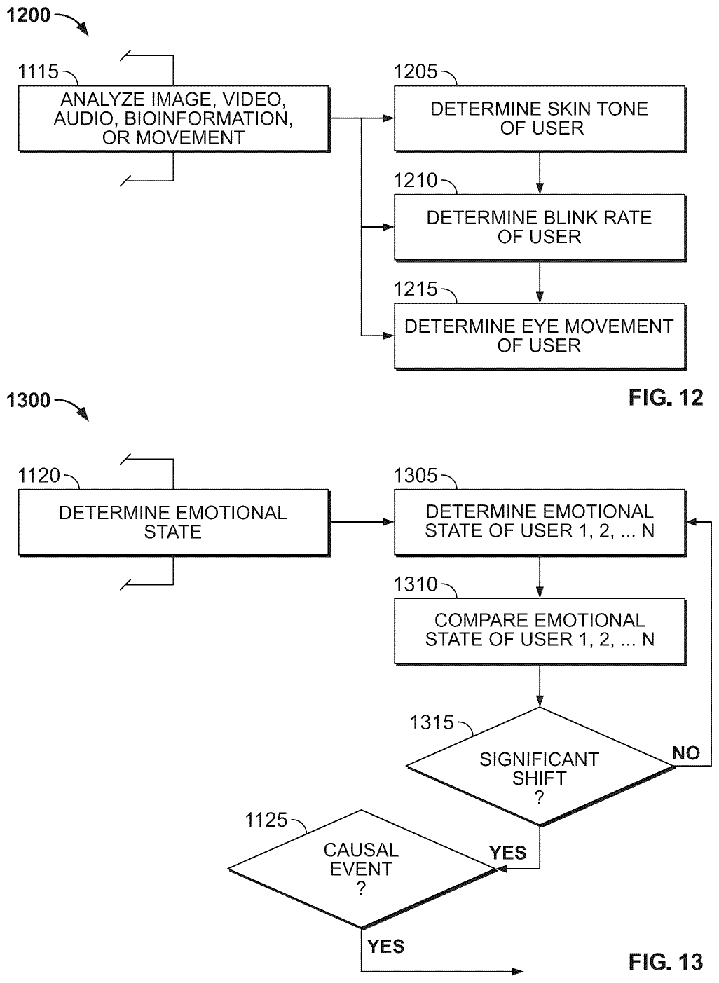

As shown in FIG. 12, the analyzing 1115 may include determining 1205 a skin tone of the user, determining 1210 a blink rate of the user, and/or determining 1215 an eye movement of the user. As shown in FIG. 13, the determining 1120 may include determining 1305 an emotional state of one or more users, comparing 1310 the emotional state of the one or more users, and, in response to determining 1315 a significant shift in the emotional state of the one or more users (“Yes” at step 1315), identifying 1125 the causal event (“Yes” at step 1125).

Artifacts and souvenirs may be generated in accordance with methods and systems for visual effects and content enhancements for XR/AR/VR/MR and 3D content and for conversion of 3D content into 2D-viewable formats. Specifically, the present specification hereby incorporates by reference herein in their entireties the full disclosures of U.S. patent application Ser. Nos. 17/975,049 and 17/975,057, both titled “VISUAL EFFECTS AND CONTENT ENHANCEMENTS FOR VR,” and both filed Oct. 27, 2022. The '049 and '057 applications are directed to, inter alia, methods and systems to enable creation of an enhanced image (e.g., 2D or 3D images, photos, videos) of a view of a VR environment. Also, the present specification hereby incorporates by reference herein in its entirety the full disclosure of U.S. patent application Ser. No. 18/086,407, titled “NATURAL AND INTERACTIVE 3D VIEWING ON 2D DISPLAYS,” filed Dec. 21, 2022. The '407 application is directed to, inter alia, methods and systems for conversion of imagery and/or video for three-dimensional (3D) displays, 4D experiences, next-gen UIs, XR, VR, AR, MR experiences, interactive experiences, and the like into imagery and/or video suitable for a two-dimensional (2D) display. In some embodiments of the '407 application, movements in the 3D environment are translated into 2D representations of the same.

### Predictive Model

Throughout the present disclosure, determinations, predictions, likelihoods, and the like are determined with one or more predictive models. For example, FIG. 16 depicts a predictive model. A prediction process 1600 includes a predictive model 1650 in some embodiments. The predictive model 1650 receives as input various forms of data about one, more or all the users, media content items, devices, and data described in the present disclosure. The predictive model 1650 performs analysis based on at least one of hard rules, learning rules, hard models, learning models, usage data, load data, analytics of the same, metadata, or profile information, and the like. The predictive model 1650 outputs one or more predictions of a future state of any of the devices described in the present disclosure. A load-increasing event is determined by load-balancing techniques, e.g., least connection, least bandwidth, round robin, server response time, weighted versions of the same, resource-based techniques, and address hashing. The predictive model 1650 is based on input including at least one of a hard rule 1605, a user-defined rule 1610, a rule defined by a content provider 1615, a hard model 1620, or a learning model 1625.

The predictive model 1650 receives as input usage data 1630. The predictive model 1650 is based, in some embodiments, on at least one of a usage pattern of the user or media device, a usage pattern of the requesting media device, a usage pattern of the media content item, a usage pattern of the communication system or network, a usage pattern of the profile, or a usage pattern of the media device.

The predictive model 1650 receives as input load-balancing data 1635. The predictive model 1650 is based on at least one of load data of the display device, load data of the requesting media device, load data of the media content item, load data of the communication system or network, load data of the profile, or load data of the media device.

The predictive model 1650 receives as input metadata 1640. The predictive model 1650 is based on at least one of metadata of the streaming service, metadata of the requesting media device, metadata of the media content item, metadata of the communication system or network, metadata of the profile, or metadata of the media device. The metadata includes information of the type represented in the media device manifest.

The predictive model 1650 is trained with data. The training data is developed in some embodiments using one or more data techniques including but not limited to data selection, data sourcing, and data synthesis. The predictive model 1650 is trained in some embodiments with one or more analytical techniques including but not limited to classification and regression trees (CART), discrete choice models, linear regression models, logistic regression, logit versus probit, multinomial logistic regression, multivariate adaptive regression splines, probit regression, regression techniques, survival or duration analysis, and time series models. The predictive model 1650 is trained in some embodiments with one or more machine learning approaches including but not limited to supervised learning, unsupervised learning, semi-supervised learning, reinforcement learning, and dimensionality reduction. The predictive model 1650 in some embodiments includes regression analysis including analysis of variance (ANOVA), linear regression, logistic regression, ridge regression, and/or time series. The predictive model 1650 in some embodiments includes classification analysis including decision trees and/or neural networks. In FIG. 16, a depiction of a multi-layer neural network is provided as a non-limiting example of a predictive model 1650, the neural network including an input layer (left side), three hidden layers (middle), and an output layer (right side) with 32 neurons and 192 edges, which is intended to be illustrative, not limiting. The predictive model 1650 is based on data engineering and/or modeling techniques. The data engineering techniques include exploration, cleaning, normalizing, feature engineering, and scaling. The modeling techniques include model selection, training, evaluation, and tuning. The predictive model 1650 is operationalized using registration, deployment, monitoring, and/or retraining techniques.

The predictive model 1640 is configured to output results to a device or multiple devices. The device includes means for performing one, more, or all the features referenced herein of the methods, processes, and outputs of one or more of FIGS. 2, 3, and 5-15, inclusive, in any suitable combination. The device is at least one of a server 1655, a tablet 1660, a media display device 1665, a network-connected computer 1670, a media device 1675, a computing device 1680, or the like.

The predictive model 1650 is configured to output a current state 1681, and/or a future state 1683, and/or a determination, a prediction, or a likelihood 1685, and the like. The current state 1681, and/or the future state 1683, and/or the determination, the prediction, or the likelihood 1685, and the like may be compared 1690 to a predetermined or determined standard. In some embodiments, the standard is satisfied (1690=OK) or rejected (1690=NOT OK). If the standard is satisfied or rejected, the predictive process 1600 outputs at least one of the current state, the future state, the determination, the prediction, or the likelihood to any device or module disclosed herein.

### Communication System

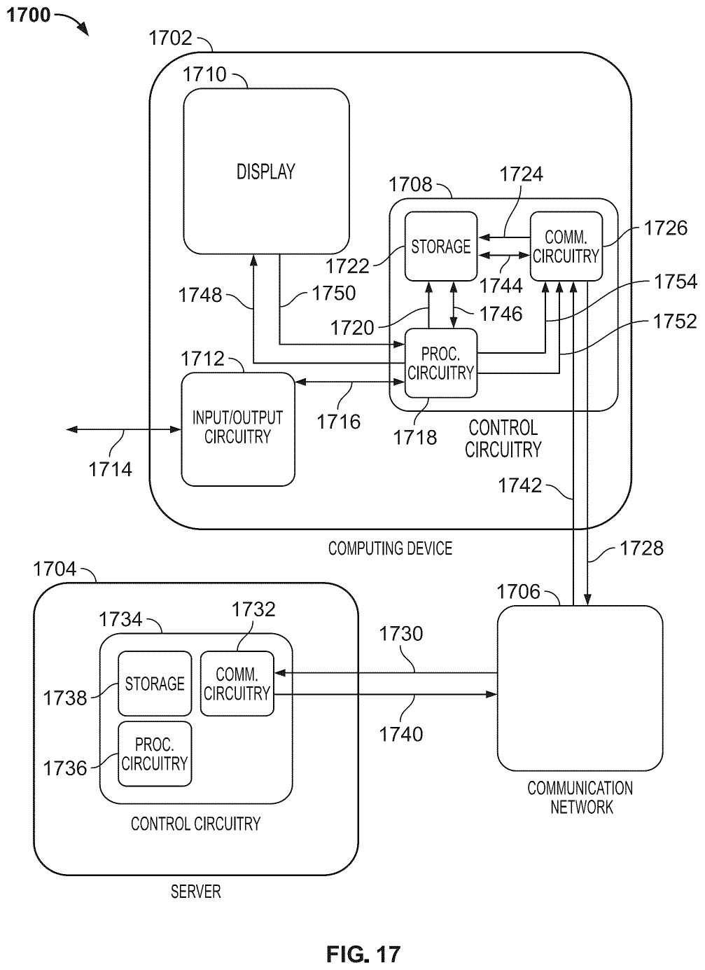

FIG. 17 depicts a block diagram of system 1700, in accordance with some embodiments. The system is shown to include computing device 1702, server 1704, and a communication network 1706. It is understood that while a single instance of a component may be shown and described relative to FIG. 17, additional embodiments of the component may be employed. For example, server 1704 may include, or may be incorporated in, more than one server. Similarly, communication network 1706 may include, or may be incorporated in, more than one communication network. Server 1704 is shown communicatively coupled to computing device 1702 through communication network 1706. While not shown in FIG. 17, server 1704 may be directly communicatively coupled to computing device 1702, for example, in a system absent or bypassing communication network 1706.

Communication network 1706 may include one or more network systems, such as, without limitation, the Internet, LAN, Wi-Fi, wireless, or other network systems suitable for audio processing applications. The system 1700 of FIG. 17 excludes server 1704, and functionality that would otherwise be implemented by server 1704 is instead implemented by other components of the system depicted by FIG. 17, such as one or more components of communication network 1706. In still other embodiments, server 1704 works in conjunction with one or more components of communication network 1706 to implement certain functionality described herein in a distributed or cooperative manner. Similarly, the system depicted by FIG. 17 excludes computing device 1702, and functionality that would otherwise be implemented by computing device 1702 is instead implemented by other components of the system depicted by FIG. 17, such as one or more components of communication network 1706 or server 1704 or a combination of the same. In other embodiments, computing device 1702 works in conjunction with one or more components of communication network 1706 or server 1704 to implement certain functionality described herein in a distributed or cooperative manner.

Computing device 1702 includes control circuitry 1708, display 1710 and input/output (I/O) circuitry 1712. Control circuitry 1708 may be based on any suitable processing circuitry and includes control circuits and memory circuits, which may be disposed on a single integrated circuit or may be discrete components. As referred to herein, processing circuitry should be understood to mean circuitry based on at least one microprocessors, microcontrollers, digital signal processors, programmable logic devices, field-programmable gate arrays (FPGAs), or application-specific integrated circuits (ASICs), etc., and may include a multi-core processor (e.g., dual-core, quad-core, hexa-core, or any suitable number of cores). In some embodiments, processing circuitry may be distributed across multiple separate processors or processing units, for example, multiple of the same type of processing units (e.g., two Intel Core i7 processors) or multiple different processors (e.g., an Intel Core i5 processor and an Intel Core i7 processor). Some control circuits may be implemented in hardware, firmware, or software. Control circuitry 1708 in turn includes communication circuitry 1726, storage 1722 and processing circuitry 1718. Either of control circuitry 1708 and 1734 may be utilized to execute or perform any or all the methods, processes, and outputs of one or more of FIGS. 1-28, or any combination of steps thereof (e.g., as enabled by processing circuitries 1718 and 1736, respectively).

In addition to control circuitry 1708 and 1734, computing device 1702 and server 1704 may each include storage (storage 1722, and storage 1738, respectively). Each of storages 1722 and 1738 may be an electronic storage device. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 8D disc recorders, digital video recorders (DVRs, sometimes called personal video recorders, or PVRs), solid state devices, quantum storage devices, gaming consoles, gaming media, or any other suitable fixed or removable storage devices, and/or any combination of the same. Each of storage 1722 and 1738 may be used to store several types of content, metadata, and/or other types of data. Non-volatile memory may also be used (e.g., to launch a boot-up routine and other instructions). Cloud-based storage may be used to supplement storages 1722 and 1738 or instead of storages 1722 and 1738. In some embodiments, a user profile and messages corresponding to a chain of communication may be stored in one or more of storages 1722 and 1738. Each of storages 1722 and 1738 may be utilized to store commands, for example, such that when each of processing circuitries 1718 and 1736, respectively, are prompted through control circuitries 1708 and 1734, respectively. Either of processing circuitries 1718 or 1736 may execute any of the methods, processes, and outputs of one or more of FIGS. 1-28, or any combination of steps thereof.

In some embodiments, control circuitry 1708 and/or 1734 executes instructions for an application stored in memory (e.g., storage 1722 and/or storage 1738). Specifically, control circuitry 1708 and/or 1734 may be instructed by the application to perform the functions discussed herein. In some embodiments, any action performed by control circuitry 1708 and/or 1734 may be based on instructions received from the application. For example, the application may be implemented as software or a set of and/or one or more executable instructions that may be stored in storage 1722 and/or 1738 and executed by control circuitry 1708 and/or 1734. The application may be a client/server application where only a client application resides on computing device 1702, and a server application resides on server 1704.

The application may be implemented using any suitable architecture. For example, it may be a stand-alone application wholly implemented on computing device 1702. In such an approach, instructions for the application are stored locally (e.g., in storage 1722), and data for use by the application is downloaded on a periodic basis (e.g., from an out-of-band feed, from an Internet resource, or using another suitable approach). Control circuitry 1708 may retrieve instructions for the application from storage 1722 and process the instructions to perform the functionality described herein. Based on the processed instructions, control circuitry 1708 may determine a type of action to perform in response to input received from I/O circuitry 1712 or from communication network 1706.

In client/server-based embodiments, control circuitry 1708 may include communication circuitry suitable for communicating with an application server (e.g., server 1704) or other networks or servers. The instructions for carrying out the functionality described herein may be stored on the application server. Communication circuitry may include a cable modem, an Ethernet card, or a wireless modem for communication with other equipment, or any other suitable communication circuitry. Such communication may involve the Internet or any other suitable communication networks or paths (e.g., communication network 1706). In another example of a client/server-based application, control circuitry 1708 runs a web browser that interprets web pages provided by a remote server (e.g., server 1704). For example, the remote server may store the instructions for the application in a storage device.

The remote server may process the stored instructions using circuitry (e.g., control circuitry 1734) and/or generate displays. Computing device 1702 may receive the displays generated by the remote server and may display the content of the displays locally via display 1710. For example, display 1710 may be utilized to present a string of characters. This way, the processing of the instructions is performed remotely (e.g., by server 1704) while the resulting displays, such as the display windows described elsewhere herein, are provided locally on computing device 1704. Computing device 1702 may receive inputs from the user via input/output circuitry 1712 and transmit those inputs to the remote server for processing and generating the corresponding displays.

Alternatively, computing device 1702 may receive inputs from the user via input/output circuitry 1712 and process and display the received inputs locally, by control circuitry 1708 and display 1710, respectively. For example, input/output circuitry 1712 may correspond to a keyboard and/or a set of and/or one or more speakers/microphones which are used to receive user inputs (e.g., input as displayed in a search bar or a display of FIG. 17 on a computing device). Input/output circuitry 1712 may also correspond to a communication link between display 1710 and control circuitry 1708 such that display 1710 updates in response to inputs received via input/output circuitry 1712 (e.g., simultaneously update what is shown in display 1710 based on inputs received by generating corresponding outputs based on instructions stored in memory via a non-transitory, computer-readable medium).

Server 1704 and computing device 1702 may transmit and receive content and data such as media content via communication network 1706. For example, server 1704 may be a media content provider, and computing device 1702 may be a smart television configured to download or stream media content, such as a live news broadcast, from server 1704. Control circuitry 1734, 1708 may send and receive commands, requests, and other suitable data through communication network 1706 using communication circuitry 1732, 1726, respectively. Alternatively, control circuitry 1734, 1708 may communicate directly with each other using communication circuitry 1732, 1726, respectively, avoiding communication network 1706.

It is understood that computing device 1702 is not limited to the embodiments and methods shown and described herein. In nonlimiting examples, computing device 1702 may be a television, a Smart TV, a set-top box, an integrated receiver decoder (IRD) for handling satellite television, a digital storage device, a digital media receiver (DMR), a digital media adapter (DMA), a streaming media device, a DVD player, a DVD recorder, a connected DVD, a local media server, a BLU-RAY player, a BLU-RAY recorder, a personal computer (PC), a laptop computer, a tablet computer, a WebTV box, a personal computer television (PC/TV), a PC media server, a PC media center, a handheld computer, a stationary telephone, a personal digital assistant (PDA), a mobile telephone, a portable video player, a portable music player, a portable gaming machine, a smartphone, or any other device, computing equipment, or wireless device, and/or combination of the same, capable of suitably displaying and manipulating media content.

Computing device 1702 receives user input 1714 at input/output circuitry 1712. For example, computing device 1702 may receive a user input such as a user swipe or user touch. It is understood that computing device 1702 is not limited to the embodiments and methods shown and described herein.

User input 1714 may be received from a user selection-capturing interface that is separate from device 1702, such as a remote-control device, trackpad, or any other suitable user movement-sensitive, audio-sensitive or capture devices, or as part of device 1702, such as a touchscreen of display 1710. Transmission of user input 1714 to computing device 1702 may be accomplished using a wired connection, such as an audio cable, USB cable, ethernet cable and the like attached to a corresponding input port at a local device, or may be accomplished using a wireless connection, such as Bluetooth, Wi-Fi, WiMAX, GSM, UTMS, CDMA, TDMA, 8G, 4G, 4G LTE, 5G, or any other suitable wireless transmission protocol. Input/output circuitry 1712 may include a physical input port such as a 12.5 mm (0.4921 inch) audio jack, RCA audio jack, USB port, ethernet port, or any other suitable connection for receiving audio over a wired connection or may include a wireless receiver configured to receive data via Bluetooth, Wi-Fi, WiMAX, GSM, UTMS, CDMA, TDMA, 3G, 4G, 4G LTE, 5G, or other wireless transmission protocols.

Processing circuitry 1718 may receive user input 1714 from input/output circuitry 1712 using communication path 1716. Processing circuitry 1718 may convert or translate the received user input 1714 that may be in the form of audio data, visual data, gestures, or movement to digital signals. In some embodiments, input/output circuitry 1712 performs the translation to digital signals. In some embodiments, processing circuitry 1718 (or processing circuitry 1736, as the case may be) carries out disclosed processes and methods.

Processing circuitry 1718 may provide requests to storage 1722 by communication path 1720. Storage 1722 may provide requested information to processing circuitry 1718 by communication path 1746. Storage 1722 may transfer a request for information to communication circuitry 1726 which may translate or encode the request for information to a format receivable by communication network 1706 before transferring the request for information by communication path 1728. Communication network 1706 may forward the translated or encoded request for information to communication circuitry 1732, by communication path 1730.

At communication circuitry 1732, the translated or encoded request for information, received through communication path 1730, is translated or decoded for processing circuitry 1736, which will provide a response to the request for information based on information available through control circuitry 1734 or storage 1738, or a combination thereof. The response to the request for information is then provided back to communication network 1706 by communication path 1740 in an encoded or translated format such that communication network 1706 forwards the encoded or translated response back to communication circuitry 1726 by communication path 1742.

At communication circuitry 1726, the encoded or translated response to the request for information may be provided directly back to processing circuitry 1718 by communication path 1754 or may be provided to storage 1722 through communication path 1744, which then provides the information to processing circuitry 1718 by communication path 1746. Processing circuitry 1718 may also provide a request for information directly to communication circuitry 1726 through communication path 1752, where storage 1722 responds to an information request (provided through communication path 1720 or 1744) by communication path 1724 or 1746 that storage 1722 does not contain information pertaining to the request from processing circuitry 1718.

Processing circuitry 1718 may process the response to the request received through communication paths 1746 or 1754 and may provide instructions to display 1710 for a notification to be provided to the users through communication path 1748. Display 1710 may incorporate a timer for providing the notification or may rely on inputs through input/output circuitry 1712 from the user, which are forwarded through processing circuitry 1718 through communication path 1748, to determine how long or in what format to provide the notification. When display 1710 determines the display has been completed, a notification may be provided to processing circuitry 1718 through communication path 1750.

The communication paths provided in FIG. 17 between computing device 1702, server 1704, communication network 1706, and all subcomponents depicted are examples and may be modified to reduce processing time or enhance processing capabilities for each step in the processes disclosed herein by one skilled in the art.

### Terminology

The terminology used herein is for the purpose of describing particular embodiments only and is not intended to be limiting of the disclosure.

Throughout the present disclosure, the term “XR” includes without limitation extended reality (XR), augmented reality (AR), 3D content, 4D experiences, next-gen UIs, virtual reality (VR), mixed reality (MR) experiences, interactive experiences, a combination of the same, and the like.

As used herein, the terms “real time,” “simultaneous,” “substantially on-demand,” and the like are understood to be nearly instantaneous but may include delay due to practical limits of the system. Such delays may be on the order of milliseconds or microseconds, depending on the application and nature of the processing. Relatively longer delays (e.g., greater than a millisecond) may result due to communication or processing delays, particularly in remote and cloud computing environments.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Although at least one embodiment is described as using a plurality of units or modules to perform a process or processes, it is understood that the process or processes may also be performed by one or a plurality of units or modules. Additionally, it is understood that the term controller/control unit may refer to a hardware device that includes a memory and a processor. The memory may be configured to store the units or the modules and the processor may be specifically configured to execute said units or modules to perform one or more processes which are described herein.

Unless specifically stated or obvious from context, as used herein, the term “about” is understood as within a range of normal tolerance in the art, for example within 2 standard deviations of the mean. “About” may be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The use of the terms “first”, “second”, “third”, and so on, herein, are provided to identify structures or operations, without describing an order of structures or operations, and, to the extent the structures or operations are used in an embodiment, the structures may be provided or the operations may be executed in a different order from the stated order unless a specific order is definitely specified in the context.

The methods and/or any instructions for performing any of the embodiments discussed herein may be encoded on computer-readable media. Computer-readable media includes any media capable of storing data. The computer-readable media may be transitory, including, but not limited to, propagating electrical or electromagnetic signals, or may be non-transitory (e.g., a non-transitory, computer-readable medium accessible by an application via control or processing circuitry from storage) including, but not limited to, volatile and non-volatile computer memory or storage devices such as a hard disk, floppy disk, USB drive, DVD, CD, media cards, register memory, processor caches, random access memory (RAM), etc.

The interfaces, processes, and analysis described may, in some embodiments, be performed by an application. The application may be loaded directly onto each device of any of the systems described or may be stored in a remote server or any memory and processing circuitry accessible to each device in the system. The generation of interfaces and analysis there-behind may be performed at a receiving device, a sending device, or some device or processor therebetween.

The systems and processes discussed herein are intended to be illustrative and not limiting. One skilled in the art would appreciate that the actions of the processes discussed herein may be omitted, modified, combined, and/or rearranged, and any additional actions may be performed without departing from the scope of the invention. More generally, the disclosure herein is meant to provide examples and is not limiting. Only the claims that follow are meant to set bounds as to what the present disclosure includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the methods and systems described herein may be performed in real time. It should also be noted that the methods and/or systems described herein may be applied to, or used in accordance with, other methods and/or systems.

This specification discloses embodiments, which include, but are not limited to, the following items:

Item 1. A method for automatically generating a digital or physical souvenir or keepsake of a user engaged in an extended reality session, the method comprising:
receiving at least one of an image of the user, a video of the user, an audio file of the user, bioinformation of the user, or a movement of the user;
analyzing the at least one of the image, the video, the audio file, the bioinformation, or the movement;
determining an emotional state of the user based on the analyzing;
identifying a causal event based on the determining;
in response to the identifying of the causal event, identifying session information of the extended reality session; and
generating the artifact based on the session information.

Item 2. The method of item 1, comprising:
training a model with at least one of the image, the video, the audio file, the bioinformation, the movement, the extended reality session, the emotional state, the causal event, the session information, or the artifact; and
during a subsequent extended reality session, performing at least one of the receiving, the analyzing, the determining, the identifying of the causal event, the identifying of the session information, or the generating based on the model.

Item 3. The method of item 2, comprising verifying the model by comparing the artifact with a quality metric.

Item 4. The method of item 2, wherein the model is trained with the image, the video, the audio file, the bioinformation, the movement, the extended reality session, the emotional state, the causal event, the session information, and the artifact.

Item 5. The method of item 1, wherein the receiving includes the image, the video, the audio file, the bioinformation, and the movement, and wherein the analyzing includes the image, the video, the audio file, the bioinformation, and the movement.

Item 6. The method of item 1, wherein the bioinformation includes at least one of a heart rate, an electroencephalogram reading, or a respiration rate.

Item 7. The method of item 5, wherein the analyzing includes determining at least one of a change in a skin tone of the user, a blink rate of the user, or an eye movement of the user.

Item 8. The method of item 1, wherein the determining of the emotional state of the user includes determining the emotional state of each user of a group of users including the user, and wherein each user of the group of users is engaged in the extended reality session.

Item 9. The method of item 8, wherein the identifying of the causal event includes comparing the emotional state of each user of the group of users engaged in the extended reality session.

Item 10. The method of item 9, wherein the causal event occurs in response to a significant shift in the emotional state of a first user of the group of users relative to the emotional state of a second user of the group of users.

Item 11. The method of item 8, wherein the extended reality session includes a collaborative game, and wherein the determining includes correlating the emotional state of a portion of the group of users, and, in response to a positive correlation of the correlated emotional state of the portion of the group of users, identifying the causal event.

Item 12. The method of item 8, wherein the extended reality session includes a competitive game, and wherein the determining includes correlating the emotional state of a portion of the group of users, and, in response to a negative correlation of the correlated emotional state of the portion of the group of users, identifying the causal event.

Item 13. The method of item 1, wherein the generating the artifact includes generating at least one of an image, a video, a three-dimensional model, a non-fungible token, a holographic image, a three-dimensional printed object, or a piece of merchandise.

Item 14. The method of item 1, comprising generating for display a user interface including a user selectable option to generate the artifact.

Item 15. The method of item 14, wherein the artifact is at least one of an image, a video, a three-dimensional model, a non-fungible token, a holographic image, a three-dimensional printed object, or a piece of merchandise.

Item 16. The method of item 1, wherein the session information includes a time-stamped emotion sequence.

Item 17. The method of item 16, wherein the time-stamped emotion sequence is correlated with the session information.

Item 18. The method of item 1, wherein the session information includes information in a scene descriptor format, and wherein the artifact includes a portion of the session information in the scene descriptor format.

Item 19. The method of item 1, wherein the session information is stored in a circular capture buffer, and wherein the artifact is generated based on the session information stored in the circular capture buffer.

Item 20. The method of item 1, comprising detecting a type of sequence during the extended reality session.

Item 21. The method of item 20, wherein the type of sequence is at least one of an action sequence, crossing a waypoint, crossing a marker, winning a competition, winning a game, or completing an action.

Item 22. The method of item 1, wherein the generating of the artifact includes changing a viewpoint of the extended reality session to include an image of the user.

Item 23. The method of item 1, comprising generating for display a user interface including a user selectable option to generate a curated list of content from the extended reality session.

Item 24. The method of item 1, comprising generating for display a user interface including user selectable options to identify conditions for generating the artifact.

Item 25. The method of item 24, wherein the conditions include a scene type, and the emotional state of the user.

Item 26. The method of item 25, wherein the scene type includes at least one of action, scenic, with others, or at a finish line, and wherein the emotional state includes at least one of happy, excited, amazed, or curious.

Item 27. The method of item 1, wherein the artifact includes a series of content items at a time before, during, and after the causal event.

Item 28. The method of item 1, comprising determining a limited or unlimited storage state of a storage device configured to store at least a portion of the extended reality session, and adjusting an amount of the session information based on the determining of the limited or unlimited storage state.

Item 29. The method of item 1, comprising receiving, from a sensor, the at least one of the image, the video, the audio file, the bioinformation, or the movement during the extended reality session.

Item 30. The method of item 29, wherein the sensor is any one of an optical sensor, an electromechanical sensor, a camera, a heart rate monitor, a blood pressure monitor, an electroencephalogram, a respiration rate monitor, a skin sensor, an eye tracking sensor, an accelerometer, a gyroscope, or a magnetometer.

Item 31. The method of item 1, comprising receiving the at least one of the image, the video, the audio file, the bioinformation, or the movement from a first sensor provided with a device configured to deliver the extended reality session to the user, and/or from a second sensor external to the device.

Item 32. The method of item 1, comprising performing at least one of the receiving, the analyzing, the determining, the identifying of the causal event, the identifying of the session information, or the generating with a processor provided with a device configured to deliver the extended reality session to the user.

Item 33. The method of item 1, comprising performing at least one of the receiving, the analyzing, the determining, the identifying of the causal event, the identifying of the session information, or the generating with a processor external to a device configured to deliver the extended reality session to the user.

Item 34. The method of item 1, comprising correlating the emotional state with the session information.

Item 35. The method of item 1, comprising determining a probability of the emotional state.

Item 36. The method of item 35, wherein the causal event occurs in response to the probability crossing a predetermined value over a period of time.

Item 37. The method of item 35, wherein the causal event occurs in response to the probability rising above a predetermined amount above a mean, mode, or median of the emotional state over a period of time.

Item 38. The method of item 1, comprising post-processing of at least one of the image, the video, the audio file, the session information, or the artifact.

Item 39. The method of item 1, comprising performing for a plurality of users at least one of the receiving, the analyzing, the determining, the identifying of the causal event, the identifying of the session information, or the generating.

Item 40. The method of item 39, comprising combining output of the at least one of the receiving, the analyzing, the determining, the identifying of the causal event, or the identifying of the session information for the generating of the artifact.

Item 41. The method of item 40, wherein the determining of the emotional state includes identifying a positive emotion or a negative emotion.

Item 42. The method of item 1, wherein the determining of the emotional state includes identifying at least one of admiration, adoration, amusement, anger, anxiety, appreciation, arousal, attention, awe, awkwardness, boredom, calmness, cognitive task engagement, compassion, confusion, contempt, contentment, craving, decision-making, depression, desire, disgust, drowsiness, embarrassment, engagement, entrancement, envy, excitement, fatigue, fear, fright, gratitude, grief, guilt, happiness, hope, horror, hunger, indifference, interest, jealousy, joy, love, memory, mental arithmetic engagement, mental workload, motor execution, nostalgia, pain, passion, pleasure, pride, rage, relief, romance, sadness, satisfaction, shame, stress, surprise, valence, or visual attention.

Item 43. The method of item 42, wherein the artifact is based on a portion of the session information including the user exhibiting the at least one of admiration, adoration, amusement, anger, anxiety, appreciation, arousal, attention, awe, awkwardness, boredom, calmness, cognitive task engagement, compassion, confusion, contempt, contentment, craving, decision-making, depression, desire, disgust, drowsiness, embarrassment, engagement, entrancement, envy, excitement, fatigue, fear, fright, gratitude, grief, guilt, happiness, hope, horror, hunger, indifference, interest, jealousy, joy, love, memory, mental arithmetic engagement, mental workload, motor execution, nostalgia, pain, passion, pleasure, pride, rage, relief, romance, sadness, satisfaction, shame, stress, surprise, valence, or visual attention.

Item 44. The method of item 1, comprising determining a demographic group of a user engaged in the extended reality session; and adjusting the generating of the artifact based on the demographic group.

Item 45. The method of item 44, wherein the demographic group includes at least one of an age group, a gender, a nationality, a language preference, a cultural group, a group membership, or a team membership.

Item 46. The method of item 1, comprising stylizing the artifact based on the emotional state.

Item 47. A method for automatically generating a digital or physical souvenir or keepsake of a user engaged in a gaming session, the method comprising:
receiving at least one of an image of the user, a video of the user, an audio file of the user, bioinformation of the user, or a movement of the user;
analyzing the at least one of the image, the video, the audio file, the bioinformation, or the movement;
determining an emotional state of the user based on the analyzing;
identifying a causal event based on the determining;
in response to the identifying of the causal event, identifying session information of the gaming session; and
generating the artifact based on the session information.

Item 48. A device configured to perform the method of any one of items 1-47.

Item 49. A device comprising means for performing the steps of the method of any one of items 1-47.

Item 50. A non-transitory, computer-readable medium having non-transitory, computer-readable instructions encoded thereon, that, when executed perform the method of any one of items 1-47.

Item 51. A system for automatically generating a digital or physical souvenir or keepsake of a user engaged in an extended reality session, the system comprising:
circuitry configured to:
   receive at least one of an image of the user, a video of the user, an audio file of the user, bioinformation of the user, or a movement of the user;
   analyze the at least one of the image, the video, the audio file, the bioinformation, or the movement;
   determine an emotional state of the user based on the analyzing;
   identify a causal event based on the determining;
   in response to the identifying of the causal event, identify session information of the extended reality session; and
   generate the artifact based on the session information.

Item 52. The system of item 51, wherein the circuitry is configured to:
train a model with at least one of the image, the video, the audio file, the bioinformation, the movement, the extended reality session, the emotional state, the causal event, the session information, or the artifact; and
during a subsequent extended reality session, perform at least one of the receiving, the analyzing, the determining, the identifying of the causal event, the identifying of the session information, or the generating based on the model.

Item 53. The system of item 52, wherein the circuitry is configured to verify the model by comparing the artifact with a quality metric.

Item 54. The system of item 52, wherein the model is trained with the image, the video, the audio file, the bioinformation, the movement, the extended reality session, the emotional state, the causal event, the session information, and the artifact.

Item 55. The system of item 51, wherein the receiving includes the image, the video, the audio file, the bioinformation, and the movement, and wherein the analyzing includes the image, the video, the audio file, the bioinformation, and the movement.

Item 56. The system of item 51, wherein the bioinformation includes at least one of a heart rate, an electroencephalogram reading, or a respiration rate.

Item 57. The system of item 55, wherein the analyzing includes determining at least one of a change in a skin tone of the user, a blink rate of the user, or an eye movement of the user.

Item 58. The system of item 51, wherein the determining of the emotional state of the user includes determining the emotional state of each user of a group of users including the user, and wherein each user of the group of users is engaged in the extended reality session.

Item 59. The system of item 58, wherein the identifying of the causal event includes comparing the emotional state of each user of the group of users engaged in the extended reality session.

Item 60. The system of item 59, wherein the causal event occurs in response to a significant shift in the emotional state of a first user of the group of users relative to the emotional state of a second user of the group of users.

Item 61. The system of item 58, wherein the extended reality session includes a collaborative game, and wherein the determining includes correlating the emotional state of a portion of the group of users, and, in response to a positive correlation of the correlated emotional state of the portion of the group of users, identifying the causal event.

Item 62. The system of item 58, wherein the extended reality session includes a competitive game, and wherein the determining includes correlating the emotional state of a portion of the group of users, and, in response to a negative correlation of the correlated emotional state of the portion of the group of users, identifying the causal event.

Item 63. The system of item 51, wherein the generating the artifact includes generating at least one of an image, a video, a three-dimensional model, a non-fungible token, a holographic image, a three-dimensional printed object, or a piece of merchandise.

Item 64. The system of item 51, wherein the circuitry is configured to generate for display a user interface including a user selectable option to generate the artifact.

Item 65. The system of item 64, wherein the artifact is at least one of an image, a video, a three-dimensional model, a non-fungible token, a holographic image, a three-dimensional printed object, or a piece of merchandise.

Item 66. The system of item 51, wherein the session information includes a time-stamped emotion sequence.

Item 67. The system of item 66, wherein the time-stamped emotion sequence is correlated with the session information.

Item 68. The system of item 51, wherein the session information includes information in a scene descriptor format, and wherein the artifact includes a portion of the session information in the scene descriptor format.

Item 69. The system of item 51, wherein the session information is stored in a circular capture buffer, and wherein the artifact is generated based on the session information stored in the circular capture buffer.

Item 70. The system of item 51, wherein the circuitry is configured to detect a type of sequence during the extended reality session.

Item 71. The system of item 70, wherein the type of sequence is at least one of an action sequence, crossing a waypoint, crossing a marker, winning a competition, winning a game, or completing an action.

Item 72. The system of item 51, wherein the generating of the artifact includes changing a viewpoint of the extended reality session to include an image of the user.

Item 73. The system of item 51, wherein the circuitry is configured to generate for display a user interface including a user selectable option to generate a curated list of content from the extended reality session.

Item 74. The system of item 51, wherein the circuitry is configured to generate for display a user interface including user selectable options to identify conditions for generating the artifact.

Item 75. The system of item 74, wherein the conditions include a scene type, and the emotional state of the user.

Item 76. The system of item 75, wherein the scene type includes at least one of action, scenic, with others, or at a finish line, and wherein the emotional state includes at least one of happy, excited, amazed, or curious.

Item 77. The system of item 51, wherein the artifact includes a series of content items at a time before, during, and after the causal event.

Item 78. The system of item 51, wherein the circuitry is configured to determine a limited or unlimited storage state of a storage device configured to store at least a portion of the extended reality session, and adjust an amount of the session information based on the determining of the limited or unlimited storage state.

Item 79. The system of item 51, wherein the circuitry is configured to receive, from a sensor, the at least one of the image, the video, the audio file, the bioinformation, or the movement during the extended reality session.

Item 80. The system of item 79, wherein the sensor is any one of an optical sensor, an electromechanical sensor, a camera, a heart rate monitor, a blood pressure monitor, an electroencephalogram, a respiration rate monitor, a skin sensor, an eye tracking sensor, an accelerometer, a gyroscope, or a magnetometer.

Item 81. The system of item 51, wherein the circuitry is configured to receive the at least one of the image, the video, the audio file, the bioinformation, or the movement from a first sensor provided with a device configured to deliver the extended reality session to the user, and/or from a second sensor external to the device.

Item 82. The system of item 51, wherein the circuitry is configured to perform at least one of the receiving, the analyzing, the determining, the identifying of the causal event, the identifying of the session information, or the generating with a processor provided with a device configured to deliver the extended reality session to the user.

Item 83. The system of item 51, wherein the circuitry is configured to perform at least one of the receiving, the analyzing, the determining, the identifying of the causal event, the identifying of the session information, or the generating with a processor external to a device configured to deliver the extended reality session to the user.

Item 84. The system of item 51, wherein the circuitry is configured to correlate the emotional state with the session information.

Item 85. The system of item 51, wherein the circuitry is configured to determine a probability of the emotional state.

Item 86. The system of item 85, wherein the causal event occurs in response to the probability crossing a predetermined value over a period of time.

Item 87. The system of item 85, wherein the causal event occurs in response to the probability rising above a predetermined amount above a mean, mode, or median of the emotional state over a period of time.

Item 88. The system of item 51, wherein the circuitry is configured to post-process at least one of the image, the video, the audio file, the session information, or the artifact.

Item 89. The system of item 51, wherein the circuitry is configured to perform for a plurality of users at least one of the receiving, the analyzing, the determining, the identifying of the causal event, the identifying of the session information, or the generating.

Item 90. The system of item 89, wherein the circuitry is configured to combine output of the at least one of the receiving, the analyzing, the determining, the identifying of the causal event, or the identifying of the session information for the generating of the artifact.

Item 91. The system of item 90, wherein the determining of the emotional state includes identifying a positive emotion or a negative emotion.

Item 92. The system of item 51, wherein the determining of the emotional state includes identifying at least one of admiration, adoration, amusement, anger, anxiety, appreciation, arousal, attention, awe, awkwardness, boredom, calmness, cognitive task engagement, compassion, confusion, contempt, contentment, craving, decision-making, depression, desire, disgust, drowsiness, embarrassment, engagement, entrancement, envy, excitement, fatigue, fear, fright, gratitude, grief, guilt, happiness, hope, horror, hunger, indifference, interest, jealousy, joy, love, memory, mental arithmetic engagement, mental workload, motor execution, nostalgia, pain, passion, pleasure, pride, rage, relief, romance, sadness, satisfaction, shame, stress, surprise, valence, or visual attention.

Item 93. The system of item 92, wherein the artifact is based on a portion of the session information including the user exhibiting the at least one of admiration, adoration, amusement, anger, anxiety, appreciation, arousal, attention, awe, awkwardness, boredom, calmness, cognitive task engagement, compassion, confusion, contempt, contentment, craving, decision-making, depression, desire, disgust, drowsiness, embarrassment, engagement, entrancement, envy, excitement, fatigue, fear, fright, gratitude, grief, guilt, happiness, hope, horror, hunger, indifference, interest, jealousy, joy, love, memory, mental arithmetic engagement, mental workload, motor execution, nostalgia, pain, passion, pleasure, pride, rage, relief, romance, sadness, satisfaction, shame, stress, surprise, valence, or visual attention.

Item 94. The system of item 51, wherein the circuitry is configured to determine a demographic group of a user engaged in the extended reality session; and adjust the generating of the artifact based on the demographic group.

Item 95. The system of item 94, wherein the demographic group includes at least one of an age group, a gender, a nationality, a language preference, a cultural group, a group membership, or a team membership.

Item 96. The system of item 51, wherein the circuitry is configured to stylize the artifact based on the emotional state.

Item 97. A system for automatically generating a digital or physical souvenir or keepsake of a user engaged in a gaming session, the system comprising:
circuitry configured to:
   receive at least one of an image of the user, a video of the user, an audio file of the user, bioinformation of the user, or a movement of the user;
   analyze the at least one of the image, the video, the audio file, the bioinformation, or the movement;
   determine an emotional state of the user based on the analyzing;
   identify a causal event based on the determining;
   in response to the identifying of the causal event, identify session information of the gaming session; and
   generate the artifact based on the session information.

Item 99. A system comprising circuitry configured to perform the method of any one of items 1-47.

While some portions of this disclosure may refer to “convention” or “conventional” examples. Any such reference is merely to provide context to the instant disclosure and does not form any admission as to what constitutes the state of the art.

Accordingly, this description is to be taken only by way of example and not to otherwise limit the scope of the embodiments herein. Therefore, it is the object of the appended claims to cover all such variations and modifications as come within the true spirit and scope of the embodiments herein.

1. A method for automatically generating a digital or physical souvenir or keepsake of a user engaged in an extended reality session, the method comprising:
receiving at least one of an image of the user, a video of the user, an audio file of the user, bioinformation of the user, or a movement of the user;
analyzing the at least one of the image, the video, the audio file, the bioinformation, or the movement;
determining an emotional state of the user based on the analyzing;
identifying a causal event based on the determining;
in response to the identifying of the causal event, identifying session information of the extended reality session; and
generating the artifact based on the session information.

2. The method of claim 1, comprising:
training a model with at least one of the image, the video, the audio file, the bioinformation, the movement, the extended reality session, the emotional state, the causal event, the session information, or the artifact; and
during a subsequent extended reality session, performing at least one of the receiving, the analyzing, the determining, the identifying of the causal event, the identifying of the session information, or the generating based on the model.

3. The method of claim 2, comprising verifying the model by comparing the artifact with a quality metric.

4. The method of claim 2, wherein the model is trained with the image, the video, the audio file, the bioinformation, the movement, the extended reality session, the emotional state, the causal event, the session information, and the artifact.

5. The method of claim 1, wherein the receiving includes the image, the video, the audio file, the bioinformation, and the movement, and wherein the analyzing includes the image, the video, the audio file, the bioinformation, and the movement.

6. The method of claim 1, wherein the bioinformation includes at least one of a heart rate, an electroencephalogram reading, or a respiration rate.

7. The method of claim 5, wherein the analyzing includes determining at least one of a change in a skin tone of the user, a blink rate of the user, or an eye movement of the user.

8. The method of claim 1, wherein the determining of the emotional state of the user includes determining the emotional state of each user of a group of users including the user, and wherein each user of the group of users is engaged in the extended reality session.

9. The method of claim 8, wherein the identifying of the causal event includes comparing the emotional state of each user of the group of users engaged in the extended reality session.

10. The method of claim 9, wherein the causal event occurs in response to a significant shift in the emotional state of a first user of the group of users relative to the emotional state of a second user of the group of users.

11.-46. (canceled)

47. A method for automatically generating a digital or physical souvenir or keepsake of a user engaged in a gaming session, the method comprising:
receiving at least one of an image of the user, a video of the user, an audio file of the user, bioinformation of the user, or a movement of the user;
analyzing the at least one of the image, the video, the audio file, the bioinformation, or the movement;
determining an emotional state of the user based on the analyzing;
identifying a causal event based on the determining;
in response to the identifying of the causal event, identifying session information of the gaming session; and
generating the artifact based on the session information.

48.-50. (canceled)

51. A system for automatically generating a digital or physical souvenir or keepsake of a user engaged in an extended reality session, the system comprising:
circuitry configured to:
receive at least one of an image of the user, a video of the user, an audio file of the user, bioinformation of the user, or a movement of the user;
analyze the at least one of the image, the video, the audio file, the bioinformation, or the movement;
determine an emotional state of the user based on the analyzing;
identify a causal event based on the determining;
in response to the identifying of the causal event, identify session information of the extended reality session; and
generate the artifact based on the session information.

52. The system of claim 51, wherein the circuitry is configured to:
train a model with at least one of the image, the video, the audio file, the bioinformation, the movement, the extended reality session, the emotional state, the causal event, the session information, or the artifact; and
during a subsequent extended reality session, perform at least one of the receiving, the analyzing, the determining, the identifying of the causal event, the identifying of the session information, or the generating based on the model.

53. The system of claim 52, wherein the circuitry is configured to verify the model by comparing the artifact with a quality metric.

54. The system of claim 52, wherein the model is trained with the image, the video, the audio file, the bioinformation, the movement, the extended reality session, the emotional state, the causal event, the session information, and the artifact.

55. The system of claim 51, wherein the receiving includes the image, the video, the audio file, the bioinformation, and the movement, and wherein the analyzing includes the image, the video, the audio file, the bioinformation, and the movement.

56. The system of claim 51, wherein the bioinformation includes at least one of a heart rate, an electroencephalogram reading, or a respiration rate.

57. The system of claim 55, wherein the analyzing includes determining at least one of a change in a skin tone of the user, a blink rate of the user, or an eye movement of the user.

58. The system of claim 51, wherein the determining of the emotional state of the user includes determining the emotional state of each user of a group of users including the user, and wherein each user of the group of users is engaged in the extended reality session.

59. The system of claim 58, wherein the identifying of the causal event includes comparing the emotional state of each user of the group of users engaged in the extended reality session.

60.-98. (canceled)
